(19) United States
(12) Patent Application Publication
(10) Pub. No.: US 2002/0069423 A1
(43) Pub. Date: Jun. 6, 2002

(54) Prion-free transgenic ungulates

(76) Inventors: Deborah J. Good, Amherst, MA (US); Jose Cibelli, Holden, MA (US)

Correspondence Address: PILLSBURY WINTHROP LLP, 1600 TYSONS BOULEVARD, MCLEAN, VA, 22102, US

(21) Appl. No.: 09/816,546

(22) Filed: Mar. 26, 2001

Related U.S. Application Data
(60) Provisional application No. 60/191,772, filed on Mar. 24, 2000.

(51) Int. Cl. A01K 67/027

(52) U.S. Cl. 800/15

(57) ABSTRACT

Transgenic and cloned ungulates and particularly cloned cattle are disclosed, wherein such cattle contain a deletion or disruption of the prion gene locus and do not express functional prion protein, and are not susceptible to prion-related diseases such as bovine spongiform encephalopy or Mad Cow Disease.

### CROSS REFERENCE TO RELATED APPLICATION

This application claims priority from U.S. Provisional Patent Application Ser. No. 60/191,772, filed Mar. 24, 2000, and is incorporated herein in its entirety.

### FIELD OF THE INVENTION

The present invention relates to transgenic and cloned ungulates and particularly cattle comprising a gene deletion or disruption, and specifically cattle having a deletion or disruption in the prion gene. Cattle that do not express prions may be unsusceptible to prion-related diseases such as bovine spongiform encephalopy (BSE), or Mad Cow Disease, and are therefor a preferred source for producing human therapeutics and other products. Creation of a line of cattle that are protected from contracting and transmitting prion-related diseases will safe-guard against the possible spread of such diseases to humans.

### BACKGROUND OF THE INVENTION

Prion-Based Diseases

Prion diseases are fatal neurodegenerative diseases that are transmittable to humans and other mammals.⁶ The most well known forms are scrapie in sheep, bovine spongiform encephalopathy (BSE) or Mad Cow Disease in cattle, and Creutzfeldt-Jakob Disease (CJD) in humans. Prior to 1987, spongiform encephalopathies were thought to be rare, and confined to sheep. By the 1990s, a growing number of cattle were afflicted with BSE, primarily in the UK. In addition, spongiform encephalopathies have been detected in zoo animals, mink, deer, and domestic cats.⁶ 6. Prusiner S. B. (1997). Prion diseases and the BSE crisis. Science 278, 245-251.

BSE was first recognized in 1986 in the United Kingdom. Now some reports state that more than 55% of cattle in the UK are infected with BSE.⁷ The rapid increase in the number of reported cases can be linked to the inclusion of infected bovine and ovine bone and meat products in food meant for cattle consumption—a sort of forced cannibalism on the part of the cattle, in the late 1970s.⁸ In controlled experiments, BSE can be transmitted to cattle, mice, sheep, goats, pigs and monkeys by either intracerebral injection or direct consumption of infected tissue.⁸⁻¹³ 7. Stekel D. J., Nowak M. A. and Southwood T. R. (1996). Prediction of future BSE spread. Nature 381, 119. 8. Taylor D. M. (1993). Bovine spongiform encephalopathy and its association with the feeding of ruminant-derived protein. Dev. Biol. Stand 80, 215-224. 9. Meldrum K. C. (1990). Transmission of BSE to a pig. Vet. Res. 127, 362. 10. Kimberlin R. H. and Walker C. A. (1989). Pathogenesis of scrapie in mice after intragastric infection. Virus Res. 12, 213-220. 11. Fraser H., Bruce M. E., Chrec A., McConnell I. and Wells G. A. (1992). Transmission of bovine spongiform encephalopathy and scrapie to mice. J. Gen. Virol 73, 1891-1897. 12. Foster J. D., Hope J. and Fraser H. (1993). Transmission of bovine spongiform encephalopathy to sheep and goats. Vet Res. 133. 13. Culip R. C., Miller J. M., Race R. E., Jenny A. L., Katz J. B., Lehnkuhl H. D., DeBey B. M. and Robinson M. M. (1994). Intracerebral transmission of scrapie to cattle. J Infect. Dis. 169, 814-829.

There are several human prion diseases which have been identified. Kuru, once seen in the Fore Highlanders of New Guinea, was characterized by loss of coordination (ataxia) and often later by dementia. It was probably passed through ritualistic cannibalism, wherein the tribe would honor the dead by eating their brains. CJD, in contrast, occurs world-wide and typically manifests itself as dementia. Most of the time it appears sporadically, striking one person in a million, typically around the age of sixty. About 10-15% of the cases are inherited, and some cases are caused inadvertently through attempts to cure other disorders. For instance, CJD has been transmitted by corneal transplantation, implantation of dura mater or electrodes in the brain, and injection of human growth hormone before it was produced recombinantly. Two other human disorders are Gerstmann-Straussler-Scheinker disease and fatal familial insomnia, both of which are usually inherited and typically appear in mid-life.⁴⁷ 47. S. Prusiner, http://www.nmia.com/˜mdibble/prion.html

In the 1990s, a variant form of Creutzfeldt-Jakob disease (vCJD) was recognized. The form of the protease resistant prion protein in this human variant is different than inherited CJD, but identical to both naturally transmitted and experimentally-induced BSE.¹⁴ Thus, it is postulated that vCJD is the result of human infection by consumption of contaminated beef or other bovine products.¹⁴ BSE has also been transmitted through ingestion of contaminated food to domestic cats.^{15,16} More than one million infected cows may have entered the food chain in the UK, suggesting that controls need to be put into place in the United States and in other countries as well to prevent the spread of this deadly disease. To date, 48 British people have died from vCJD and there is new evidence that this variant form of CJD and BSE are one in the same. 14. Collinge J., Sidle K. C. L., Meads J., konside J. and Hill A. F. (1996). Molecular analysis of prion strain variation and the aetiology of new variant CJD. Nature 383, 685-690. 15. Truyen U., Parrish C. R., Harder T. C. and Kaaden O. R. (1995). There is nothing permanent except change. The emergence of new virus diseases. Vet. Microbiol. 43, 103-112. 16. Pearson G. R., Wyatt J. M., Gruffydd-Jones T. J., Hope J., Chong A., Higgins R. J., Scott A. C. and Wells G. A. (1992). Feline spongiform encephalopathy; fibril and PrP studies. Vet. Rec. 131, 307-310.

The PrP Gene and Protein

The infectious agent of BSE and other prion-based diseases is a cellular protein named PrP. PrP is a cell membrane-associated glycoprotein expressed in the central and peripheral nervous Systems.^{17,18} In₁₃ scrapie, BSE and CJD, the normally protease-sensitive PrP protein becomes protease-resistant. This apparently occurs through a change in protein conformation whereby the normal cellular form consisting primarily of alpha helices changes into the disease form consisting mainly of beta sheets.⁴⁷ This change in conformation may occur more readily with certain PrP mutations. For instance, in one inherited form of human CJD, Pro¹⁰² is mutated to Leu. When this mutation is introduced into transgenic mice, these animals develop CNS degeneration and amyloid-like PrP plaques.¹⁹⁻²¹ 17. Fournier K. G., Escaig-Haye F., Billette de Villemeur T. and Robain 0. (1995). Ultrastructural localization of cellular prion protein (PrPc) in synaptic boutons of normal hamster hippocampus. C. R. Acad. Sci. All318, 339-334. 18. DeArmond S. J., Mobley W. C., DeMott D. L., Barry R. A., Beckstead J. H. and Prusiner S. B. (1987). Changes in the localization of brain prion protein during scrapie infection. Neurology 37, 1271-1280. 19. Hsiao K. K., Scott M., Foster D., Groth D. F., DeArmond S. J. and Prusiner S. B. (1990). Spontaneous neurodegeneration in transgenic mice with mutant prion protein. Science 250, 1587-1590. 20. Hsiao K., Scott M., Foster D., DeArmond S.J., Groth D., Serban H. and Prusiner S. B. (1991). Spontaneous neurodegeneration in transgenic mice with prion protein codon 101 proline—leucine substitution. Ann NY Acad Sci 640, 166-170. 21. Hsiao K. K., Groth D., Scott M., Yang S. L., Serban H., Rapp D., Foster D., Torchia M., Dearmond S. J. and Prusiner S. B. (1994). Serial transmission in rodents of neurodegeneration from transgenic mice expressing mutant prion protein. Proc. Nat'l Acad. Sci. USA 91, 9126-9130. 47. S. Prusiner, http://www.nmia.com/˜mdibble/prion.html

It is not exactly clear how one or all cellular prions suddenly switch conformation, but one hypothesis is that a disease prion having a beta sheet conformation somehow induces the alpha helical prions to also change to a beta sheet conformation. For instance, it has been shown that when cellular and scrapie prions are mixed together in a test tube, cellular prions are converted into scrapie prions.⁴⁷ It has been postulated that some mutations in the prion gene render the resulting proteins more susceptible to flipping into the beta sheet conformation. Presumably it takes time until one molecule spontaneously flips and still more time for disease prions to accumulate and damage the brain enough to cause symptoms.⁴⁷ Alternatively, there may be other factors or proteins which influence the likelihood of conversion. For instance, it has been shown that certain bacterial and yeast chaperone proteins make the conversion to the beta sheet form much more efficient.⁴⁸ 47. S. Prusiner, http://www.nmia.com/˜mdibble/prion.html 48. Proc. Natl. Acad. Sci. USA 94:13; New Scientist, Science, 17 May, p.16

The gene for PrP, called PRNP is located on chromosome 20 in humans.^{22,23} The gene consists of three exons, with an mRNA of approximately 2.4 kb in humans. The third exon of PRNP contains the entire protein coding domain and encodes a 25 kDa protein. Twenty different mutations in the human PRNP gene have been found in inherited prion diseases.⁶ In sporadic CJD, no coding mutations in the PrP protein have been found, but all patients are homozygous for a methionine residue at position 129.¹⁴ This may indicate that this polymorphism predisposes to infection with certain prion strains. Prion particles from the new variant strain of CJD (vCJD) has been shown to have a glycosylation pattern different than other human prion isoforms, but similar to BSE prions, and like sporadic CJD, no protein coding mutation at residue position 129. These data are consistent with the hypothesis that the new variant strain of CJD arose from BSE transmission to humans. 6. Prusiner S. B. (1997). Prion diseases and the BSE crisis. Science 278, 245-251. 14. Collinge J., Sidle K. C. L., Meads J., konside J. and Hill A. F. (1996). Molecular analysis of prion strain variation and the aetiology of new variant CJD. Nature 383, 685-690. 22. Liao Y. C., Lebo R. V., Clawson G. A. and Smuckler E. A. (1986). Human prion protein cDNA; molecular cloning, chromosomal mapping and biological implications. Science 233, 364-367. 23. Sparkes R. S., Simon M., Cohn V. H., Fournier R. E., Lem J., Klisak I., Heinzmann C., Blatt C., Lucero M. and Mohandas T. (1986). Assignment of the human and mouse prion protein genes to homologous chromosomes. Proc. Natl. Acad Sci. USA 83, 7358-7362.

Prevention and Control of Prion-Based Diseases

There is no known cure for any of the prion-based encephalopathies. Thus, guidelines to control the spread of the disease in both livestock and humans have been put into place by the World Health Organization and other national and international coalitions. In 1988, all bovine material was banned from food meant for cattle consumption.²⁴ By 1989, the Spongiformn Encephalopathy Advisory Committee (SEAC) recommended that the brain, spleen, thymus, tonsil, and gut should be discarded from all cattle, and that clinically ill cattle should be incinerated. Unfortunately, according to SEAC, these guidelines were put in place too late to prevent the spread of BSE from infected meat products to humans.⁵ 5. (1995). Transmissible Spongiform Encephalopathies: A summary of present knowledge and research. Spongiform Encephalopathy Advisory Committee, London. 24. Anderson R. M., Donnelly C. A., Ferguson N. M., Woolhouse M. E. J., Watt C. J., Udy H. J., MaWhinney S., Dunstan S. P., Southwood T. R. E., Wilesmith J. W., Rayns J. B. M ., Hoinville L. J., Hillerton J. E. Austin A. R. and Wells G. A. H. (1996). Transmission dynamics and epidemiology of BSE in British cattle. Nature 382, 779-788.

Only one drug has been shown to control the onset of neurodegeneration by prions in an animal. In a controlled study using amphotericin B, this agent delayed the accumulation of PrP^{ac} in scrapie-infected hamsters.²⁵ Other compounds, including pentosan polysulfate and Congo red prevent accumulation of PrP^{ac} in cell culture, but have not been tested in animal models.²⁶ 25. Xi Y. G., Ingrosso L., Ladogana A., Masullo C. and Pocchiari M. (1992). Amphotericin B treatment dissociates in vivo replication of the scrapie agent from PrP accumulation. Nature 356, 598-601. 26. Caughey B. and Raymond G. J. (1993). Sulfated polyanion inhibition of scrapie-associated PrP accumulation in cultured cells. J Virol 67, 643-650.

Research in the field of prevention and control of prion-based diseases has shown that one copy of a normal PRNP gene is necessary for both susceptibility to and transmission of the disease. Mice containing a targeted deletion of both copies of the PRNP gene are resistant to intracerebral inoculation of scrapie prions.^{27,49-51} Thus, the disease requires synthesis of endogenous prions for accumulation of enough disease prions to result in neurodegenerative symptoms. The PrP knockout mice have normal behavior, normal development and can reproduce, suggesting that PrP is not necessary for viability or fertility.²⁷ These data suggest that creation of animals lacking the PRNP gene may halt the spread of prion-based diseases from livestock to humans and other animals. 27. Bueler H., Fisher M., Lang Y., Bluethmann H., Lipp H.-P., DeArmond S. J., Prusiner S. B., Agent M. and Weissmann C. (1992). Normal development and behavior of mice lacking the neuronal cell surface PrP protein. Nature 356, 577-582. 49. Bueler R., Aguzzi A., Sailer A., Greiner R. A., Autenried P., Aguet M. and Weissman C. (1993). Mice devoid of PrP are resistant to scrapie. Cell 73, 1339-1347. 50. Prusiner S. B., Groth D., Serban A., Koehler R., Foster D., Torchia M., Burton D., Yang S. L. and DeArmond S. J. (1993). Ablation of the prion protein (PrP) gene in mice prevents scrapie and facilitates production of anti-PrP antibodies. Proc. Natl. Acad. Sci. USA 90,10608-10612. 51. Weissman C, Bueler R, Fischer, M, Sailer A., Aguzzi A. and Aguet M. (1994). Prp-deficient mice are resistant to scrapie. Ann. N. Y Acad, Sci., 724,235-240

There is no known cure for Bovine Spongiform Encephalitis (BSE) or the human equivalent, Creutzfeldt-Jakob disease (CJD). An altered form of the prion (PrP) gene and an endogenous PrP gene are necessary for infection. It is widely accepted that infected cows transmit the disease to humans as CJD. A murine model demonstrated that ablation of the PrP gene prevents scrapie. We seek to eradicate the susceptibility to BSE in genetically modified cattle. We propose to clone a calf that contains a targeted deletion of the PrP gene. Specific aims I) design and construct a gene targeting vector. II) use this vector to carry out homologous recombination in bovine fetal fibroblasts and identify gene targeted cells with null-mutation on one allele of PrP gene. III) generate PrP heterozygous knockout (KO) bovine fetuses by nuclear transfer using gene-targeted cells generated from aim II. IV) genotype cloned fetuses and isolate PrP heterozyous KO fetal fibroblasts. V) carry out homologous recombination in PrP heterozyous KO fetal fibroblasts, and identify gene targeted cells with null-mutations on both alleles of the PrP gene. VI) generate a PrP homozygous KO bovine calf. Prion-free transgenic cattle will be used as sources of pharmaceutical, cosmetic, human therapeutics, and food products.

### SUMMARY OF THE INVENTION

The present invention discloses the first transgenic cattle to have a gene deletion. In particular, the invention encompasses transgenic and cloned ungulates containing a deletion or disruption in the endogenous prion gene, in either one or both chromosomes, such that the ungulates have less susceptibility or no susceptibility to prion-based diseases such as scrapie and bovine spongiform encephalitis (BSE). Generally, the deletions are engineered by homologously recombining a heterologous DNA into the prion gene locus such that all or part of the protein codon region is replaced or deleted. The ungulates of the present invention may in addition have a heterologous transgene which is extraneous to the prion locus for the purpose of producing therapeutic recombinant proteins, facilitating xenotransplantation of tissue, and studying prion-based diseases.

### BRIEF DESCRIPTION OF THE DRAWINGS

FIG. 1. Diagram showing the putative structure of the bovine PrP gene based on Accession numbers D26150 and D26151.⁴⁰ The prion gene in other animals and humans is composed of three exons with the entire coding region being contained within the third exon. 40. Inoue S., Tanaka M., Horiuchi M., Ishiguro N. and Shinagawa M. (1997). Characterization of the bovine prion protein gene: the expression requires interaction between the promoter and intron. J Vet. Med. Sci. 59, 175-183.

FIG. 2. (A) Structure of proposed targeting vectors 1-4. Each of these targeting vectors uses part of intron 1 and exon 2 for the 5′ flanking region and the untranslated region of exon 3 for the 3′ flanking region. All of intron 2 and all of the protein coding region of exon 3 has been deleted. (B) Structure of proposed targeting vectors 5-8. Each of these targeting vectors uses part of intron 2 and part of exon 3, including the exon 3 splice acceptor site for the 5′ flanking region. The 3′ flanking region is the same as in vectors 1-4 and contains only the 3′ untranslated region of exon 3. Most of the protein coding region of exon 3 has be deleted, leaving only 3 amino acids of the protein present.

FIG. 3. Expression of prion mRNA in bovine embryonic fibroblast (BEF) cells. Ten micrograms of RNA was isolated from BEF cells and GT1-7 cells, a hypothalamic transformed cell line, run on a formaldehyde agarose gel, transferred to a nitrocellulose membrane and probed with a 1 kb Sst fragment from the human PrP cDNA. The ethidium bromide stained ribosomal RNAs confirm that each sample was equally loaded.

FIG. 4. Southern analysis of bovine PRNP gene. Equal amounts of BEF genomic DNA was digested with the indicated enzyme, separated on an 0.8% agarose gel, transferred to nitrocellulose membrane and probed with human PRNP cDNA.

FIG. 5 shows a targeting vector and structure of recombined PrP gene.

FIG. 6 shows PCR products used for cloning of PrP.

FIG. 7 shows cell survival in electroporation of BFF cells transfected with pPNT and pPRP.

FIG. 8 shows the results of another experiment wherein BFF cells were transfected with pPNT.

FIG. 9 shows electroporation of BFF cells with pPRP.

FIG. 10 shows G418 treatment of untransfected BFF cells.

FIG. 11 shows G418 treatment of BFF cells transfected with pPNT.

FIG. 12 shows genomic DNA organization of bovine PrP and depicts schematically the gene targeting strategy. The top panel shows that the bovine PrP gene is composed of three exons and two introns, spanning over 20 Kb region [40]. Exon 1 and 2 which are 53 bp and 98 bp, respectively, are transcribed as 5′ UTR, and the Exon 3 contains sequence of 10 bp 5′ UTR, 795 bp coding region and about 3.3 Kb 3′ UTR. Intron 1 and 2 are about 2.4 Kb and 14 Kb in site. The middle parnel shows that the targeting vector contains a part of the intron 2 sequence (at least 7 Kb, exon 3 in which the base PrP coding sequence is completely deleted and replaced with a promoterless neomycin resistant gene, and partial downstream genomic sequence of exon 3. The expression of the neomycin resistance gene is under the control of the endogenous PrP promoter and its regulating elements. The bottom panel shows the targeted bovine PrP allele after homologous recombination. The shaded boxes are exons; open boxes contain names of genes with the ATP start color identified.

### DETAILED DESCRIPTION OF THE INVENTION

The present invention concerns transgenic ungulates and particularly bovines comprising a targeted gene deletion. In particular, the invention relates to transgenic ungulates bearing a either a homozygous or heterozygous deletion or disruption of the prion gene. For transgenic cattle bearing a homozygous deletion or disruption, the deletion or disruption prevents expression of a functional endogenous prion protein, wherein lack of expression of a functional endogenous prion protein renders said cattle unsusceptible to prion-related diseases. For transgenic cattle which are heterozygous for the prion deletion or disruption, the deletion or disruption renders said cattle less susceptible to prion related diseases due to decreased expression of the prion protein. In particular, said cattle are unsusceptible or less susceptible to bovine spongiform encephalitis (BSE), or Mad Cow Disease. However, deletion or disruption of the prion gene will render the animals unsusceptible or less susceptible, respectively, to any prion-related disease.

The prion deletions or disruptions of the present invention are preferably created by homologous recombination of heterologous DNA into the prion gene locus. Said heterologous DNA preferably comprises a selectable marker to facilitate identification and isolation of cells which contain the deletion or disruption. However, any heterologous DNA may be used for homologous recombination. Likewise, a second heterologous DNA may be exchanged for the selectable marker after selection and isolation of cells containing the deletion or disruption using homologous recombination. Alternatively, the heterologous DNA may be excluded or deleted after homologous recombinant cells are generated.

Where the heterologous DNA comprises a selectable marker, it is preferably a neomycin resistance gene. Said selectable marker may be operably linked to a promoter which functions in bovine cells, such as the PGK promoter. Alternatively, the selectable gene may initially be promoterless if the targeting construct for creating the deletion or disruption recombines into the prion gene locus such that the selectable marker gene is expressed from the prion gene promoter.

The transgenic ungulates of the present invention may also contain a heterologous gene that is extraneous to the prion gene locus. For instance, a second heterologous gene may be operably linked to a mammary-specific promoter, thereby enabling the production of a heterologous protein in the milk of the transgenic ungulate. For bovines, this is a convenient way of producing recombinant therapeutic proteins for the treatment of human diseases, which would have the added advantage that the bovines used to make the proteins are prion-free, thereby reducing the risk of transmission of spongiform encephalopies. Accordingly, the present invention also comprises a method of using such transgenic female bovines for the production of recombinant proteins.

Another example of a second heterologous gene which could be introduced into the ungulates of the present invention is a mutant prion gene. Several alleles of prion genes from various species have been identified which confer an increased susceptibility to prion-related diseases. Ungulates of the present invention which have a homozygous deletion or disruption of the endogenous prion gene and which are transgenic for such mutant alleles provide an ideal vehicle for studying the progression of prion-related disease in such animals without interference from prions encoded by other alleles of the gene. Moreover, developing a cloned line of such transgenic ungulates introduces the additional advantage of having an isogenic background, which is particularly ideal for studying complex disease processes where other proteins could conceivably be involved.

Accordingly, the present invention also encompasses cloned transgenic ungulates having the same genotype. Techniques for cloning cattle using nuclear transfer techniques have been discussed in detail in U.S. Pat. Nos. 5,945,577 and 6,147,276, herein incorporated by reference. The cloned transgenic ungulates may also bear a heterologous gene that is extraneous to the prion gene locus.

Developing a cloned line of transgenic mammals has advantages that surpass creating an isogenic background for the study of disease progression. Such techniques allow the production of several animals simultaneously; the techniques allow for sex selection of the founder animals; and the need for entire generations of animals may be surpassed, thereby expediting creation of a transgenic line. Accordingly, the cloned transgenic bovines of the present invention encompass every variation of ungulate described herein.

Also in this regard, the present invention encompasses not just one cloned transgenic bovine, but relates to “lines” of transgenic bovines all having the same genotype. Just as it is advantagous to have a line of cells which are each genetically identical, so is it advantageous to have a line of mammals which are genetically identically, for reliability, uniformity, etc. Imagine trying to study the affects of a reagent on a population of cells which are all genetically distinct. Having a uniform population of cells enables one to make reasoned predictions and valid conclusions concerning an entire population of mammals, without having to factor in the effects of genetic diversity.

Thus, the present invention encompasses a method of using transgenic ungulates, and particularly cloned transgenic ungulates, containing a homozygous deletion or disruption in the endogenous prion gene and a heterologous mutant prion gene, to screen for or evaluate agents which may be used in the treatment or prevention of spongiform encephalopathies. Such a method comprises (1) administering a putative therapeutic agent to said transgenic ungulate before or after the development of said prion-related spongiform encephalopy; and (2) monitoring said ungulate to determine whether the relevant prion-related spongiform encephalopy has been prevented or treated. Agents to be screened might encompass antisense nucleic acids, chemicals, antibodies or other protein ligands which inhibit either expression of the mutant prion gene, the initial conversion of cellular prions to disease-specific prions, or the conversion of cellular prions through interaction with disease-specific prions.

The transgenic ungulates of the present invention also find use as a source of tissues and cells for xenotransplantation. For instance, these animals could be used as a source of fetal neurons to treat both Parkinson's and Huntington's Disease. One product for Parkinson's disease has already demonstrated proof of principle in a pre-clinical model. This research has shown that fetal neurons from cloned cattle can be grafted into the Parkinsonian rat reversing Parkinson's disease symptoms.²⁸ It may then be possible to use transgenic bovine neurons to treat human neurodegenerative diseases. Fetal neurons can be implanted into the diseased brain of these human patients resulting in some relief of symptoms.²⁹⁻³¹ One of the controversies in this field is in the use of human fetuses for transplantation. Likewise, transplantation of human corneas, and dura matter graphs have resulted in infectious CJD in more than 60 humans.⁶ To avoid the possibility of transmissible spongiforn encephalopathies, the fetal tissues used for transplantation should come from PrP-free ungulate fetuses. 6. Prusiner S. B. (1997). Prion diseases and the BSE crisis. Science 278, 245-251. 28. Zawada W. M., Cibelli J. B., Choi P. K., Clarkson E. D., Golueke P. J., Witta S. E., Bell K. P., Kane J., Ponce de Leon F. A., Jerry J. D., Robl J. M., Freed C. R. and Stice S. L. (1998). Somatic cell cloning-produced transgenic bovine neurons for transportation in Parkinsonian rats, submitted. 29. Chisholm A. H. (1996). Fetal tissue transplantation for the treatment of Parkinson's disease: a review of the literature. J Neurosci. Nurs. 28, 329-338. 30. Defer G. L., Gerry C., Ricolfi F., Fenelon G., Monfort J. C., Remy P., Villafane G., Jeny R, Samson Y., Keravel Y., Gaston A., Degos J. D., Peschanski M., Cesaro P, and Nguyeen J.P. (1996). Long-term outcome of unilaterally transplanted Parkinsonian patients. I. Clinical approach. Brian 119, 41-50. 31. Shannon K. M. and Kordower J. H. (1996). Neural transplantation for Huntington's disease: experimental rationale and recommendations for clinical trials. Cell Transplant 5, 339-352.

Accordingly, the present invention encompasses a method of xenotransplantation using fetal tissue or cells derived from the transgenic ungulates, said method comprising (1) generating a transgenic fetus with a homozygous deletion or disruption in the endogenous prion gene, either by mating or cloning techniques; (2) isolating tissue or cells of interest from said fetus; and (3) transplanting the fetal tissue or cells into a recipient mammal. Preferably, the cells are fetal neuron cells which are used to treat either Parkinson's or Huntington's disease. Alternatively, fetal corneal tissue may be used to replace a damaged human cornea. The transgenic ungulates used as a source of tissue may also comprise a heterologous DNA, or a second gene deletion or disruption, which acts to deter transplant rejection.

In this regard, the present invention also encompasses transgenic ungulates bearing at least one other deletion or disruption that is extraneous to the prion gene locus. Such animals may also comprise a heterologous DNA extraneous to the prion disruption, and are particularly useful in the context of ungulates transgenic for mutant prion genes in that such mammals may be used to study the affects of other gene deletions on prion-related disease processes.

Prion-free cattle can also be used to increase the safety profile of bovine-derived products such as Bovine Serum Albumin (used as a carrier in many human medications, and in research laboratories) and Fetal Calf Serum (for cell culture in laboratories). Furthermore, prion-free cattle could be used by the agriculture industry to ensure safe meat products to consumers, livestock and domestic animals. Now that the methodology to create transgenic cattle has resulted in several live-born transgenic calves, it would be advantageous to supplement that technology by creating future transgenic lines that are unable to transmit, or contract BSE.

Also encompassed in the present invention are the nucleic acid constructs used to isolate and characterize an ungulate prion gene, and particularly the bovine prion gene, as well as those used to construct the targeting DNA molecule, the targeting constructs and plasmid derivatives. Specifically, the present invention encompasses an isolated DNA molecule comprising at least part of the bovine prion gene promoter operably linked to a selectable marker gene coding region or a reporter gene coding region. The phrase “at least part of the bovine promoter” indicates that the DNA molecule contains a sufficient amount of the promoter region to facilitate homologous recombination when included in a targeting construct comprising a second bovine DNA sequence from or adjacent to the bovine prion gene locus. However, also included are DNA molecules containing functional portions of the promoter operably linked to a selectable marker or reporter gene, which may be used for the purpose of monitoring transcription from the prion gene promoter in vivo or in vitro, for example, in response to transcriptional or translational regulatory mechanisms.

Preferably the selectable marker is a neomycin resistance gene. Targeting constructs may also contain a thymidine kinase gene to enable both positive and negative selection of homologous recombinants. Also encompassed are plasmid vectors comprising the isolated DNA molecule of the invention, wherein a preferred plasmid vector is one having a pUC backbone such as pBluescript (Stratagene) or pCR-Topo II (Invitrogen).

More generally, the present invention encompasses a DNA targeting molecule capable of specifically and functionally deleting or disrupting expression of an ungulate prion gene, wherein said disruption occurs by homologous recombination into the prion gene locus. In this case, one arm of the targeting construct need not necessarily be the prion gene promoter, so long as the targeting construct results in homologous recombinants unable to express the endogenous gene. Indeed, the targeting molecule may encompass a selectable marker gene operably linked to any promoter capable of functioning in ungulate or bovine cells, but preferably the PGK promoter is used. These molecules may also optionally contain a thymidine kinase gene for negative selection of cells which incorporate the targeting molecule by means other than homologous recombination. Because the entire coding region of the gene is contained within exon 3, targeting molecules of the present invention preferably facilitates the deletion or disruption of at least exon 3 of the prion gene. Plasmid vectors comprising the DNA targeting molecules are also encompassed.

Also encompassed in the present invention are methods of making the transgenic ungulates using the targeting DNA molecules of the present invention. Specifically, a method of making a transgenic ungulate heterozygous for the prion gene deletion or disruption comprises the steps of: (1) isolating genomic DNA from ungulate cells; (2) isolating a prion gene allele from said genomic DNA; (3) determining a restriction enzyme map and the intron/ exon structure of the ungulate prion gene allele isolated from said bovine genomic DNA; (4) sub-cloning fragments from said prion gene allele for the construction of a targeting DNA molecule; (5) constructing a targeting DNA molecule which is capable of disrupting or deleting a prion gene allele by homologous recombination; (6) transfecting said ungulate cells such that homologous recombinants are isolated; (7) transferring the nuclei from a transfected cell containing the targeting molecule homologously recombined into a prion gene allele to the cytoplasm of an enucleated mature ungulate oocyte; (8) culturing said oocyte to form a blastocyst; and (9) transferring said blastocyst to a recipient mammal such that a transgenic ungulate according to the invention is born. Homozygous transgenic ungulates are then obtained by breeding the heterogenous transgenic ungulates, or by targeting a deletion of the other allele using primary fibroblasts. Alternatively, a homozygous deletion or disruption may be isolated in the initial fibroblast cell.

It is noted that the method of making the transgenic ungulate is most readily accomplished using nuclear transfer techniques due to the fact that homologous recombinants may be isolated using a cell line that is easy to propagate, then the nuclei of these recombinants may then be readily transferred to an enucleated oocyte. However, it should be apparent that transgenic mammals may also be made by the standard technique of transfecting the targeting construct directly into embryonic stem cells.

The cells used for isolating genomic DNA and the prion gene locus are generally primary fibroblast cells. For the transgenic cattle of the invention, these cells are preferably derived from fetal fibroblast cells such as BEF cells. However, the cells which are used for isolation of the genomic DNA and generation of the donor nuclei may also be adult fibroblast cells, the feasibility of which has been demonstrated in U.S. Pat. No. 5,945,577, herein incorporated by reference.

Definitions

The term ungulate encompasses horses, cattle, sheep, goats, deer, and any other hoofed mammal.

The phrase “disruption” means that the deleted portion of the prion gene may be replaced with heterologous DNA such that the gene is disrupted, while “deletion” encompasses deletions which do not accompany an insertion of heterologous DNA. While deletions of the present invention need not encompass the entire prion gene, the deletions or disruptions are engineered such that no functional prion protein is expressed, and no aberrant variant protein is produced, i.e., a truncation. In fact, Shmerling et al. (1998) prepared knockout mice expressing PrPs with amino-proximal deletions and found that certain truncated derivatives caused severe ataxia and death as early as one to three months after birth.¹ 1. Scott et al (1999), Proc. Nat'l. Acad. Sci. USA, 96:15137-15142.

The term “prion-related diseases” encompasses scrapie, bovine spongiform encephalopy, or Mad Cow Disease, and any other variety of prion-based neurodegenerative disease to which ungulates are susceptible. This includes any cross-species disorders which are caused by exposure of ungulates to infectious prions from any other mammal or human.

The phrase “selectable marker” generally means any gene which by its expression enables specific selection of cells which express the gene over cells which do not. However, the term may also included markers which are screened, i.e., by visual screening assays, color indicator assays, or the like, so long as the use of said marker in combination with the transfection protocol enables identification and selection of homologous recombinants.

The phrase “extraneous to the prion gene locus” merely means that the secondary heterologous DNA is unrelated to and unnecessary for the knockout at the prion locus. However, because said heterologous DNA exists and is expressed independently, it may also be located within the homologously recombined region so long as it does not disrupt with selection of homologous recombinants, expression of the selectable marker, etc.

The phrase “operably linked” means that the DNA fragments are linked or connected in such a way that expression of one is dependent on the functioning of the other.

The phrase “derived from” means originating from and does not encompass any derivation which departs from the spirit and crux of the invention.

The scope of the present invention is illustrated by the following exemplary experiments.

Experimental Overview

The present invention involves isolation of the bovine PrP gene (PRNP), construction of the targeting vector, transfection of the donor cells, nuclear transfer of the donor nucleus to an enucleated oocyte, and transfer of the oocyte to a recipient mother. Nuclear transfer techniques are described in detail in U.S. Pat. No. 5,945,577, which is herein incorporated by reference. The remaining techniques involve the following:

1. Cloning and Characterization of the Bovine PrP Gene

The primary fibroblast cells used herein (BEF cells) have been used previously to create cloned transgenic cattle.⁴ Genomic DNA has been isolated from these cells, and used to make a BEF genomic library. The intron/exon structure of the isogenic (BEF) PrP gene (PRNP) is determined, based on putative bovine PrP structure as predicted from the sequence of other bovine PRNP genes.² 2. Schmerling et al. (1998), Cell 93:203-214. 4. Cibelli J. B., Stice S. L., Golueke P. J., Kane J. J., Jerry D. J., Blackwell C., Ponce de Leon F. A. and Robl J. M. (1998). Cloned transgenic calves produced from non-quiescent fetal fibroblasts. Science, accepted.

2. Construction of Targeting Vectors for the Bovine PrP Gene

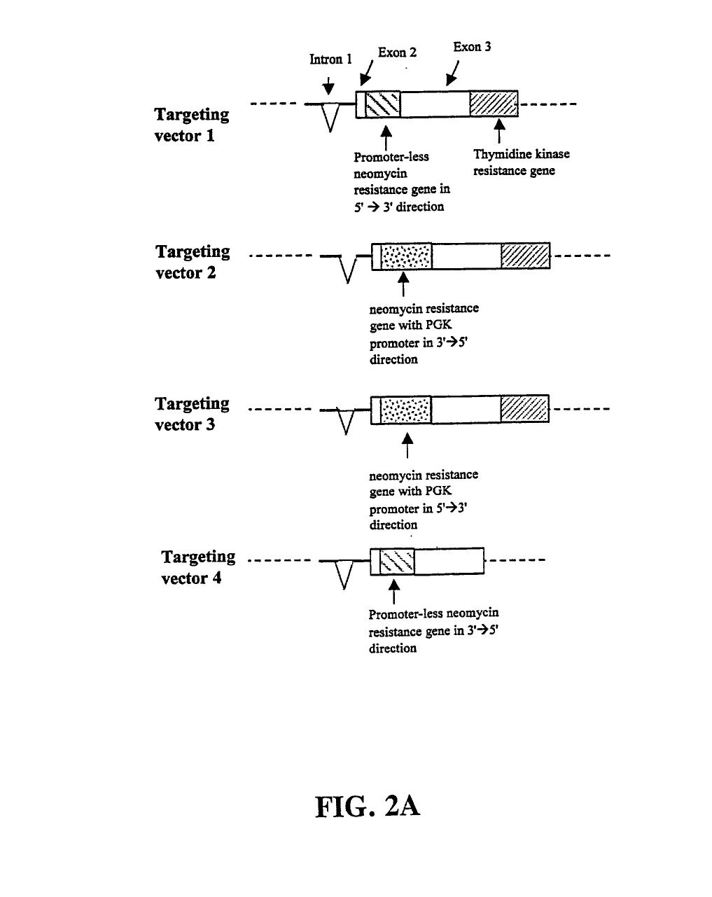

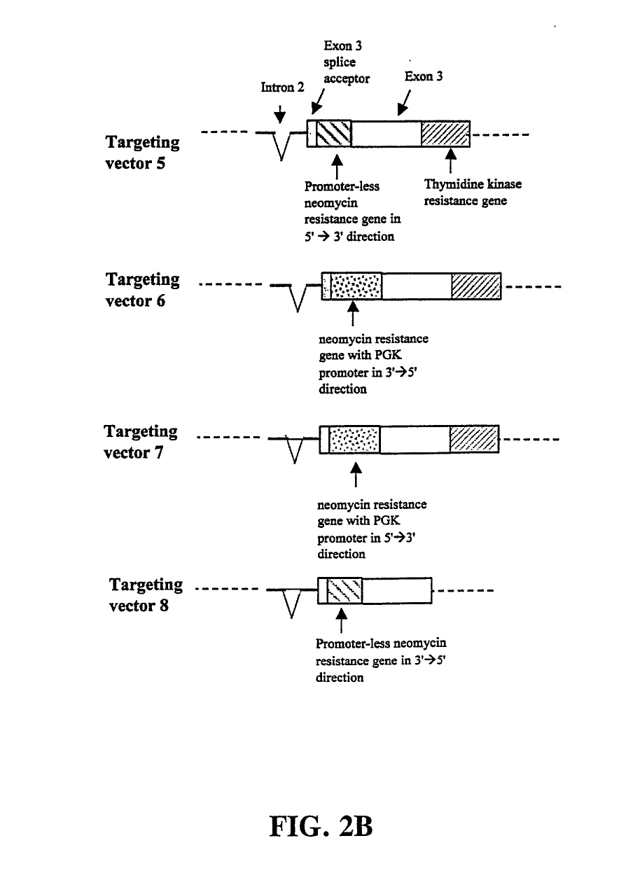

Eight different targeting vectors for the PrP gene are proposed. Vectors 2, 3, 6 and 7 are positive-negative selection targeting vectors containing a positive selectable marker (neomycin) driven by the PGK promoter, and a negative selectable marker (thymidine kinase) driven by the herpes simplex virus (HSV) promoter, along with flanking DNA from the isogenic bovine PRNP gene (see FIG. 2A and 2B). Targeting vectors 1 and 5 contain thymidine kinase as a negative selectable marker driven by the HSV promoter and a promoterless positive selectable marker driven by the HSV promoter and a promoterless positive selectable marker (neomycin) along with flanking DNA from the isogenic bovine PRNP gene. Correct integration of the targeting vector results in transcription of neo driven by the endogenous PrP promoter. Targeting vectors 4 and 8 enable only positive selection, and contain a promoterless positive selectable marker (neomycin) along with flanking DNA from the isogenic bovine PRNP gene. Expression of the neo gene is driven by the endogenous bovine PRNP promoter.

3. Optimization of Targeting Efficiency

The optimal conditions for drug selection and electroporation are determined using both a control vector and the final targeting vector. Given the low rate of homologous recombination in normal diploid cells, this is a necessary step to ensure high transfection efficiency and effective drug selection conditions to isolate rare cells containing a targeted deletion of PRNP.

Experimental Methods

Extended (Long) Polymerase Chain Reaction

The PRNP gene is amplified from BEF genomic DNA using the primer sets shown in Table 1, and the EXPANDÔ 20 kB Plus PCR system (Boehringer Mannheim) according to manufacturers' instructions. Amplified DNA is subcloned into pCR-XL-Topo II vector using the PCR cloning kit (Invitrogen).

TABLE 1
Primers used for cloning the bovine PRNP gene.

| Primer | | Position |
|---|---|---|
| Name | Sequence | in PRNP |
| A | 5′-GCA GAG CTG AGA CGC TCT TC-3′ | Exon 1 |
| B | 5′-CAG CTC AAG TTG GAT TTG TGT C-3′ | Exon 2 |
| C | 5′-GTT CAT AGA CCC AGG GTC CAC C-3′ | Exon 3 |
| D | 5′-CAG TGC ACG CTG TAA GGC TAA G-3′ | Exon 3 |
| PrP1s | 5′-GGG CAA CCT TCC TGT TTT CAT TAT C-3′ | Exon 3 |
| PrP1a | 5′-CCA TAC ACT GCA CAA ATA CAT TTT CGC-3′ | Exon 3 |
| PrP3a | 5′-CAT AAT GAA AAC AGG AAG GTT GCC C-3′ | Exon 3 |
| PrP3b | 5′-GCG AAA ATG TAT TTG TGC AGT GTA TGG-3′ | Exon 3 |
| PrP2a | 5′-GAC ACA AAT CCA ACT TGA GCT G-3′ | Exon 2 |
| PrP3c | 5′-CAC CAT GAT GAC TTA TCT GC-3′ | Exon 3 |
| PrP3d | 5′-GAA CCA GGA TCC AAC TGC CTA TG-3′ | Exon 3 |

Library Screening and Hybridization

Phage DNA is hybridized to a 2.5 kb EcoRI³² P-random-prime labeled probe of the fall length human PrP cDNA (ATCC)³⁷ using standard techniques.³⁶ 32. Izraeli S., Lowe L., Bertness V., Good D. J., Kirsch I. and Kuehn M. (1998). Requirement for the immediate-early gene SIL for body axis development and Sonic Hedgehog function in mouse embryos, in preparation. 36. Sambrook J., Fritsch E. F. and Maniatis T. (1989). “Molecular Cloning: A Laboratory Manual.” Cold Spring Harbor Laboratory Press, Cold Spring Harbor. 37. Puckett C., Concannon P., Casey C. and Hood L. (1991). Genomic structure of the human prion protein gene. Am. J Hum. Genet. 49, 871-872.

Phage Preparation and Phage DNA Purification

To prepare phage DNA, phage purification preps are used (Promega). Those kits reduce the time of phage DNA purification from one day to one hour, are reasonable in cost, and eliminate the toxic phenol/chloroform extractions of the traditional method.

Bovine Fibroblast Production, Maintenance and Electroporation

Bovine fibroblast cells (BEF) were produced from a 55-day-old Holstein male fetus according to standard fetal fibroblast preparation methods.³⁸ A large number of cells from this single fetus were prepared and have been successfully used in the past to create cloned transgenic cattle. Fibroblasts are maintained in polystyrene tissue culture plates at 37° C., 5% CO₂. Cells are passed 1:10 when they reach 80% confluency. These primary cells have a 28-30 hour cell cycle and undergo approximately thirty population doublings before senescence. 38. Love P. E., Trembley M. L. and Westphal H. (1992). Targeting of the T-cell receptor zeta chain in embryonic stem cells: strategies for generating multiple mutations in a single gene. Proc. Natl Acad. Sci. USA 89, 9929-9933.

Actively growing cells (80% confluency) are used for electroporation. The cells are harvested by trypsinization, and resuspended at a density of 5×10⁶ cells/500 μl of ice cold PBS. A 500 μl aliquot of cells is placed into an electroeluation cuvette to which 20 μg of DNA in sterile water is added. The cells and DNA are gently mixed by tapping and incubated on ice for 10 minutes. Following the ten minute incubation the cells are again gently resuspended and then electroporated with the parameters given in Table 1. Optimal parameters will be determined from these experiments. Following the pulse the cuvettes are again incubated on ice for an additional ten minutes. Under sterile conditions, we remove the cells from the cuvette, resuspended in 10 ml of the above media, and plated onto 10, 100 mm² polystyrene tissue culture dishes in a total of 10 ml of media. The cells are incubated overnight at 37° C., 5% C0₂.

Bovine Cells and DNA

The bovine PRNP DNA used to make the targeting construct should be derived from the same cells which will be transfected. In murine genomic targeting experiments, replacement vectors made with isogenic DNA (genes cloned from the same species/strain as the cells which will be targeted) increases the effective targeting rate by 2.5-fold.³⁹ Unlike mice, there are no inbred strains of cattle, and thus, the PrP gene must be cloned from the exact fetal cells that will be used in the targeting experiments to increase the frequency of recombination. Thus, a genomic library was constructed using BEF genomic DNA. The λ FIX II library was chosen because it accepts large fragments of DNA (9-23 kb) and has multiple flanking restriction enzyme sites for sub-cloning and manipulation of the cloned DNA fragments. 39. van Deursen J. and Wieringa B. (1992). Targeting of the creatine kinase M gene in embryonic stem cells using isogenic and nonisogenic vectors. Nucl. Acids Res. 20, 3815-3820.

Identification of the Bovine PrP Gene

One million plaque forming units (pfu) are plated with the host bacteria strain XL-1 blue (Stratagene) and allowed to grow for 12-16, or until lysed plaques appear. Phage particles are transferred to nitrocellulose filters, hybridized with a 2.5 kb EcoRI fragment containing the full length human PrP cDNA (ATCC, cat # 65946³⁷ ) using standard molecular biology techniques.³⁶ The human PrP cDNA shares an approximately 80% homology with the bovine gene (Blast search comparison using Accession number AB001468, bovine PrP cDNA). 36. Sambrook J., Fritsch E. F. and Maniatis T. (1989). “Molecular Cloning: A Laboratory Manual.” Cold Spring Harbor Laboratory Press, Cold Spring Harbor. 37. Puckett C., Concannon P., Casey C. and Hood L. (1991). Genomic structure of the human prion protein gene. Am. J Hum. Genet. 49, 871-872.

Positive plaques from the first round of cloning are picked, re-plated and re-hybridized to the human prion probe. Positive plaques from the third round of cloning are amplified in liquid media, and purified as described in the methods sections. Phage DNA will be purified as described in the methods section of this proposal.

Structural Characterization of the Bovine Prion Gene

A non-isogenic bovine PrP gene (meaning, not derived from BEF cells) has already been cloned from Bos taurus and the putative map is available through Gen Bank (Accession #s D26150, D26151).⁴⁰ Most of the mapping of the isogenic bovine PrP gene can be done with simple restriction enzyme digests of a phage(s) and hybridization of these digests with the different exons of the human PrP cDNA. As explained above, mapping the intron/exon structure of the bovine PrP gene from the cells to be targeted is necessary in order to achieve optimal recombination frequency using the targeting vector. It is recommended that the targeting vector disrupt expression of or delete exon 3 which contains all of the protein coding region of the gene. Thus, it is necessary to map the exact location and restriction endonuclease map of exon 3 within the isogenic bovine PrP gene. 40. Inoue S., Tanaka M., Horiuchi M., Ishiguro N. and Shinagawa M. (1997). Characterization of the bovine prion protein gene: the expression requires interaction between the promoter and intron. J Vet. Med. Sci. 59, 175-183.

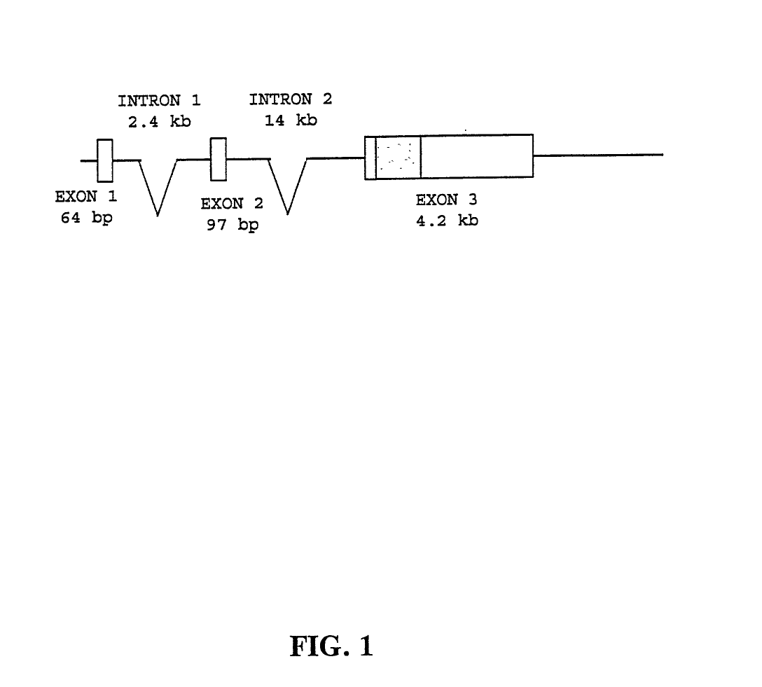

FIG. 1 shows the putative map of the bovine PrP gene based on Accession numbers D26150 and D26151.⁴⁰ The prion gene in other animals, and humans is composed of three exons with the third exon containing the entire coding region of the PrP protein. Various combinations of probes from exons 1-3 and restriction digests may be used to map the size of the introns and exons of the isogenic bovine PrP gene. 40. Inoue S., Tanaka M., Horiuchi M., Ishiguro N. and Shinagawa M. (1997). Characterization of the bovine prion protein gene: the expression requires interaction between the promoter and intron. J Vet. Med. Sci. 59, 175-183.

Restriction endonuclease digestion and mapping will reveal convenient areas for sub-cloning the PrP gene into plasmid vectors. These stretches of DNA will be purified, and ligated into the pBluescript plasmid (Stratagene). These plasmids can be used for sequence analysis of exons and for more fine mapping of individual areas of the PrP gene.

Sequence Analysis of the Isogenic Bovine PrP Gene

Once the positions of the three exons are mapped, short stretches of these areas may be sequenced to confirm their identity. The sequence data allows one to define the area of the gene and to recognize any differences between phage clones isolated from the genomic library which might signal allelic differences.

To sequence the prion gene, sub-cloned fragments of the PrP gene containing exons 1-3 are sequenced using ABI's fluorescent dRhodamine sequencing kit and either universal primers to the plasmid vector, or designed primers to internal regions. Sequence results are used to confirm the positional mapping of the exon/intron structure of the bovine PrP gene before beginning the targeting vector. It was for us of utmost importance for this targeting construct that the exact deletion be known, and confirmed for two reasons. First, the cattle of the present invention were to be the first cattle containing a targeted deletion of any gene. We wanted to be able to confirm the exact location of the deletion within the genome, and be able to exactly map the deletion in any offspring from these cattle. Second, because of the nature of the gene we are deleting, it is necessary to be confident that the sequences being deleted contain the coding region for the prion protein, and that all protein coding regions are eliminated in the resultant cattle produced by this technique.

Construction of the Targeting Vector

The Positive-Negative Type Targeting Vector

The most frequently used selectable marker gene is the neomycin resistance gene, or “neo”. This gene will confer resistance to G418 to the cells that carry a targeting construct. The thymidine kinase gene will be used to allow for negative selection in the presence of gancyclovir. This will allow us to select against cells with non-homologous insertion of the targeting vector. In murine embryonic stem cells, double selection in the presence of G418 and gancyclovir results in a 200-fold enrichment of homologous recombinants over G418 selection alone.⁴¹ It has been reported that double selection in human diploid fibroblasts results in only a 2-3 fold enrichment in homologous recombinants.⁴² For reasons that will be discussed below, we feel that even this modest increase is useful to the ultimate success of the targeting experiments. 41. Capecchi M. R. (1989). The new mouse genetics: Altering the genome by gene targeting. Trends Genet 5, 70-76. 42. Brown J. P., Wei W. and Sedivy J. M. (1997). Bypass of senescence after disruption of p. 21 cip l/wafl gene in normal diploid human fibroblasts. Science 277, 831-834.

The final design of the targeting vector depends on the restriction analysis. Since the PrP protein coding region itself is entirely contained in exon 3, the targeting vector should be constructed so as to delete or disrupt all of the protein coding region of this gene. Elimination of protein coding regions in mice successfully eliminated prion infection and transmission.^{27,49-51} A diagram of a typical targeting vector according to the invention along with the resultant PrP gene structure following targeting vector insertion is shown in FIG. 2. 27. Bueler H., Fisher M., Lang Y., Bluethmann H., Lipp H.-P., DeArmond S. J., Prusiner S. B., Agent M. and Weissmann C. (1992). Normal development and behavior of mice lacking the neuronal cell surface PrP protein. Nature 356, 577-582. 49. Bueler R., Aguzzi A., Sailer A., Greiner R. A., Autenried P., Aguet M. and Weissman C. (1993). Mice devoid of PrP are resistant to scrapie. Cell 73, 1339-1347. 50. Prusiner S. B., Groth D., Serban A., Koehler R., Foster D., Torchia M., Burton D., Yang S. L. and DeArmond S. J. (1993). Ablation of the prion protein (PrP) gene in mice prevents scrapie and facilitates production of anti-PrP antibodies. Proc. Natl. Acad. Sci. USA 90,10608-10612. 51. Weissman C, Bueler R, Fischer, M, Sailer A., Aguzzi A. and Aguet M. (1994). Prp-deficient mice are resistant to scrapie. Ann. N. Y Acad, Sci., 724,235-240

Capecchi has shown that for efficient targeting in embryonic stem cells, the vector must have flanking homologous DNA sequences of at least 1 kb in length. A two fold increase in homologous sequences resulted in a 20-fold increase in targeting frequency of the hprt locus.⁴¹ Therefore, at least 1 kb of homologous sequence on either side of the targeted deletion is recommended, most likely from non-coding regions of the PrP gene on either side of the neomycin gene. 41. Capecchi M. R. (1989). The new mouse genetics: Altering the genome by gene targeting. Trends Genet 5, 70-76.

The neo gene of the present invention was derived from the pPNT plasmid (generous gift from Dr. Heiner Westphal)⁴³ and is driven by the PGK promoter. The TK gene is from the HSV-TK plasmid.⁴⁴ Both the PGK-neo gene and HSV-TK gene have been sub-cloned into pBluescript-SK (Stratagene) to create additional cloning sites (Good, unpublished). The plasmid backbone for the entire targeting construct is the pBluescript-SK vector (Stratagene). The constraints of restriction enzyme sites and fragment sizes within the PRNP gene determine the ultimate flank size, deletion size and regions of the PRNP gene used. Our experience suggests that it is often beneficial to create two different targeting vectors to two different regions of a gene. 43. Tybulewicz, V. L., Crawford C. E., Jackson P. K., Bronson R. T. and Mulligan R. C. (1991). Neonatal lethality and lymphopenia in mice with a homozygous disruption of the c-abl proto-oncogene. Cell 65, 1153-1163. 44. Mansour S. L., Thomas K. R. and Capecchi M. R. (1988). Disruption of the proto-oncogene int-2 in mouse embryo-derived stem cells; a general strategy for targeting mutations to non-selectable genes. Nature 336, 348-352.

The Promoter-Less Neo Gene Targeting Vector

A successful targeting experiment using normal non-rodent diploid cells was reported three years ago in the lab of Dr. John Sedivy.⁴² This targeting experiment employed a promoter-less neo targeting vector, which was constructed in such a way as to be driven by the promoter of the endogenous targeted gene, when properly inserted. This technique apparently resulted in a 100-200 fold enrichment in homologous recombinants. 42. Brown J. P., Wei W. and Sedivy J. M. (1997). Bypass of senescence after disruption of p. 21 cip l/wafl gene in normal diploid human fibroblasts. Science 277, 831-834.

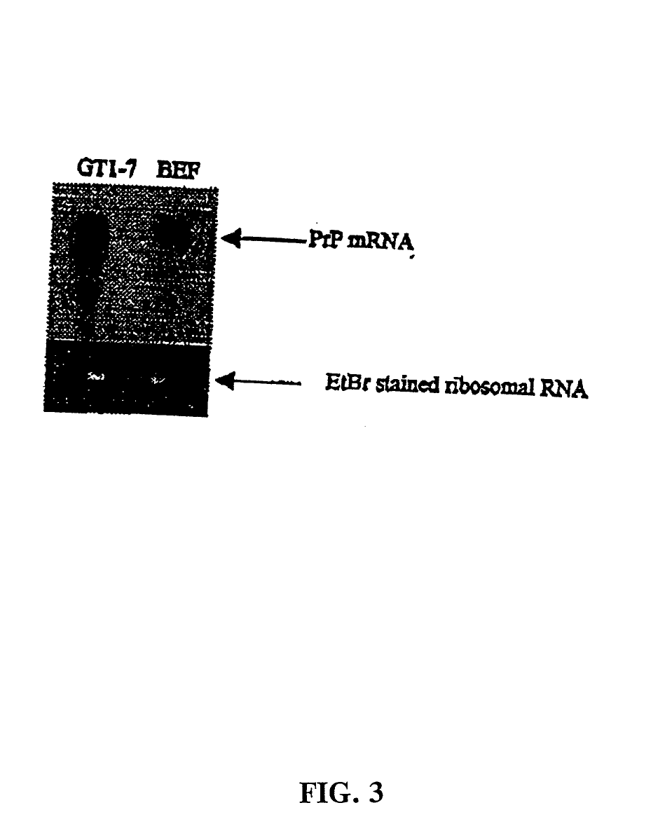

One concern with using this method was that high PrP endogenous PrP expression would be necessary to achieve sufficient neomycin expression. PrP is expressed in murine embryonic fibroblast cells.⁴⁵ Northern analysis on RNA isolated from BEF cells demonstrates that PrP mRNA is present in these cells as well (FIG. 3). This level is approximately 50% lower than the hypothalamic cell line GT1-7, but appears to be sufficient to support a promoter-less construct.

Diagrams of several promoter-less neo constructs are shown in FIGS. 2A and 2B. The final design of the targeting vector depends on the restriction analysis of the bovine PrP gene. A new neomycin cassette plasmid, containing the promoter-less neomycin gene was created using PCR-amplification of pGEM-neo-poly A plasmid. A primer recognizing the 5′ end of the neomycin gene was designed (Tk-Bam: 5′-GCC AAT ATG GGA TCG GCC ATT GAA C-3′) to be used along with the T7 promoter vector primer in a standard PCR amplification procedure. The 1.4 kb fragment was subcloned into the PCR-Topo II vector. To assure that the neomycin cassette will be placed in frame in the PrP protein, including the ATG codon, a promoter-less neomycin resistance gene is sub-cloned from the pNEO vector (Pharmacia Biotech), leaving a splice site 5′ to the neo gene, within the third exon of PRNP. Flanking DNA sequences of at least one kilobase is inserted on either side of the neo cassette, and a TK gene for negative selection in the presence of gancyclovir is inserted at the 3′ end of the construct.

Using a promoter-less neo construct is advantageous to the goal of creating a targeted deletion within the PRNP gene in that only correct integration of this construct into the PRNP gene results in synthesis of the neo resistance gene, and resistance of G418. In addition, the TK gene will be lost in correctly targeted vectors, resulting in resistance to gancyclovir. The majority of G418 resistance/TK resistant colonies in a targeting experiment, using a traditional positive-negative targeting vector, result from random insertion in the genome. The promoter-less type of construct allows only those random integrations near an active promoter to be resistant to G418. Therefore, because there will be fewer total G418 colonies present, a promoter-less targeting vector allows one to screen every G4 18 positive colony in an experiment, rather than 200 randomly selected colonies, as is done in ES cell targeting. In human diploid fibroblasts, for instance, this strategy resulted in 20 G418 resistant colonies, and four homologous recombinants—a targeting frequency of 20%.⁴² 42. Brown J. P., Wei W. and Sedivy J. M. (1997). Bypass of senescence after disruption of p. 21 cip l/wafl gene in normal diploid human fibroblasts. Science 277, 831-834.

Optimization of Targeting Efficiency in BEF Cells

Determining the Optimal Electroporation Conditions for BEF Cells

Several reports have indicated that, with the exception of embryonic stem cells, homologous recombination in most normal mammalian cells is low.^{42,46} Although BEF cells have been electroporated and transgenic cattle created in our laboratory,⁴ it is necessary to optimize the transfection efficiency in order to obtain the rare homologous recombinant containing a disruption or deletion of the prion gene. 4. Cibelli J. B., Stice S. L., Golueke P. J., Kane J. J., Jerry D. J., Blackwell C., Ponce de Leon F. A. and Robl J. M. (1998). Cloned transgenic calves produced from non-quiescent fetal fibroblasts. Science, accepted. 42. Brown J. P., Wei W. and Sedivy J. M. (1997). Bypass of senescence after disruption of p. 21 cip l/wafl gene in normal diploid human fibroblasts. Science 277, 831-834. 46. Hanson K. D. and Sedivy J. M. (1995). Analysis of biological selections for high-efficiency gene targeting. Mol. Cell. Biol. 15, 45-51.

To affect optimization of transfection, BEF cells are grown to sub-confluency, trypsinized and re-suspended in 0.5 ml of Ca⁺²/Mg⁺² free PBS along with 20 μg of linearized pPNT vector or without DNA. This plasmid contains a mutated neomycin resistance gene under the control of the phosphoglycerol kinase promoter (PGK).⁴³ The BEF cells are electroporated using the conditions listed in Table 1, and then plated as described in the methods at a density of 5×10⁵ cells per 100 mm² tissue culture plate. The values listed are those pre-set on an Invitrogen electroporation apparatus. 43. Tybulewicz, V. L., Crawford C. E., Jackson P. K., Bronson R. T. and Mulligan R. C. (1991). Neonatal lethality and lymphopenia in mice with a homozygous disruption of the c-abl proto-oncogene. Cell 65, 1153-1163.

TABLE 2
Optimization of electroporation conditions for BEF cells.

| DNA | VOLTAGE | CAPACITANCE |
|---|---|---|
| none | 0 V | 0 μF |
| none | 330 V | 1000 μF |
| none | 330 V | 500 μF |
| none | 600 V | 250 μF |
| none | 1500 V | 71 μF |
| none | 1800 V | 50 μF |
| pPNT | 0 V | 0 μF |
| pPNT | 330 V | 1000 μF |
| pPNT | 330 V | 500 μF |
| pPNT | 600 V | 250 μF |
| pPNT | 1500 V | 71 μF |
| pPNT | 1800 V | 50 μF |

The experiment should be done twice, with six plates of electroporated cells for each point. All six plates are grown overnight in drug-free media, at 37° C. and 5% CO₂. In the morning, three plates are trypsinzied, and counted to determine cell survival. The media in the remaining three plates is changed into G418-containing media (400 μg/ml). The media in these plates is changed each morning to keep the level drugs constant. After five to ten days, when visible colonies are present on the plates, the plates are stained with methylene blue and the number of colonies on each plate counted. The average of the three plates is used to determine transfection efficiency for each electroporation condition. Each electroporation condition is tried in two separate experiments.

These experiments are designed to determine the maximum DNA transfection parameters for BEF cells with the maximum cell survival. These values should be optimized before beginning homologous recombination experiments to increase the efficiency of finding these rare events in the population of transfected cells. A reasonable goal is to increase the transfection efficiency to achieve at least 1000-1500 G418 resistant colonies with each transfection (1000-1500 G418 resistant clones/3×10⁶ transfected cells=1 transfected cell in every 3000 cells). We estimated that even with a relatively low homologous targeting frequency of one homologous recombinant in every 1000 resistant cells, that we should find 2-4 homologous recombinants in a transfected population of 1×10⁷ cells: 1×10⁷ transfected cells=3333 G418 resistant cells=3.3 homologous recombinants 1 resistant cell in 3000 1 homologous recombinant per electroporation in 1000 resistant cells

This number (3.3 homologous recombinants per electroporation) is equivalent to a targeting frequency of one in three million transfected cells, and is thirty-fold lower than the frequency in human diploid fibroblasts.⁴² Thus, even if the frequency of homologous recombination is lower in BEF cells than in normal human fibroblasts, these conditions allow us to recover at least one to two targeted cells in each experiment. 42. Brown J. P., Wei W. and Sedivy J. M. (1997). Bypass of senescence after disruption of p. 21 cip l/wafl gene in normal diploid human fibroblasts. Science 277, 831-834.

Once the optimal parameters are determined using the pPNT vector, these parameters should be used to optimize the targeting frequency of the targeting vector. Since the targeting vector may be larger than the test plasmid, this may effect the transfection efficiency.

Determining Effective Drug Concentrations in BEF Cells

Before starting the actual targeting experiment, it is imperative to determine the highest concentration of neomycin (G418) and gancyclovir that is necessary for complete killing of non-transfected cells, but that will not be toxic to correctly targeted cells. Previous work in our laboratory has determined that 400 μg/ml neomycin will sufficiently kill non-neomycin containing BEF cells, but will allow the rapid proliferation of BEF cells containing a transfected neomycin gene.⁴ Because the positive-negative selection targeting that we are proposing requires that we select for several days in the presence of both gancyclovir and G418, we set up a selection curve to test the range of gancyclovir in combination with G418 that can be used in these experiments. 4. Cibelli J. B., Stice S. L., Golueke P. J., Kane J. J., Jerry D. J., Blackwell C., Ponce de Leon F. A. and Robl J. M. (1998). Cloned transgenic calves produced from non-quiescent fetal fibroblasts. Science, accepted.

In addition, work from others has shown that with human and rat diploid fibroblasts, efficient selection is only achieved when the amount of G418 added to the culture is in the range of 1-10 mg/ml.^{42,46} This is up to 10 fold higher than is normally used on BEF cells. Thus, one should first determine if higher levels of drug can be used in these cells to increase the frequency of homologous recombinants.

To answer this question, untransfected BEF cells are plated at a density 5×10⁵ cells in 10 ml of media, and incubated overnight. The following morning, the media is changed to media containing the drugs and concentrations listed in Table 2. Two plates of cells are used for each concentration of drug. G418 selection is continued for up to 10 days, or until there is complete killing of non-transfected cells on each plate. Gancyclovir selection continues for 4 days and the percent survival is calculated. 42. Brown J. P., Wei W. and Sedivy J. M. (1997). Bypass of senescence after disruption of p. 21 cip l/wafl gene in normal diploid human fibroblasts. Science 277, 831-834. 46. Hanson K. D. and Sedivy J. M. (1995). Analysis of biological selections for high-efficiency gene targeting. Mol. Cell. Biol. 15, 45-51.

TABLE 3
G418 and gancyclovir treatment of untransfected BEF cells.

| | | GANCYCLOVIR | |
|---|---|---|---|
| VECTOR | CONCENTRATION | CONCENTRATION | PURPOSE |
| None | 300 μg/ml | None | Normal cell killing |
| | | | by G418 |
| None | 1000 μg/ml | None | Normal cell killing |
| | | | by G418 |
| None | 3000 μg/ml | None | Normal cell killing |
| | | | by G418 |
| None | None | 1 μM | Normal cell |
| | | | survival in |
| | | | gancyclovir |
| None | None | 3 μM | Normal cell |
| | | | survival in |
| | | | gancyclovir |
| None | None | 10 μM | Normal cell |
| | | | survival in |
| | | | gancyclovir |

The TK gene converts gancyclovir into a toxic nucleotide analogue. Normal cells, and cells containing a correctly targeted PrP gene lack should be resistant to this drug. Since to our knowledge, this drug has not been used on BEF cells, these assays allow us to determine the highest level of gancyclovir to which the cells may be subjected without substantial toxicity in the absence of the TK gene. Optimization of both the neomycin sensitivity and gancyclovir resistance of BEF cells should increase the targeting frequency and allow one to find rare homologous recombinants in the pool of transfectants.

Selection of Transfected BEF Cells

Cells are electroporated using the optimal conditions determined above, with the pPNT vector which contains both a neomycin resistance gene as well as thymidine kinase gene.⁴³ Following the electroporation, the cells are plated at a density of 5×10⁵ cells in 10 ml of media and incubated overnight. The following morning, the media is changed to media containing the drugs and concentrations listed in Table 3. Two plates of cells are used for each concentration of drug. 43. Tybulewicz, V. L., Crawford C. E., Jackson P. K., Bronson R. T. and Mulligan R. C. (1991). Neonatal lethality and lymphopenia in mice with a homozygous disruption of the c-abl proto-oncogene. Cell 65, 1153-1163.

Gancyclovir selection alone or in combination with G418 will continue for four days with the culture medium changed once every twenty-four hours to assure high drug concentration in the media at all times. After four days, the media is changed to G418 alone, or no drug according to the chart, and selection is continued for an additional six days, or until individual colonies are visible. At that time, the media is removed from the plates, the plates rinsed once in PBS, and cell colonies stained with methylene blue. The number of colonies on each plate is counted and cell death/growth curves for each drug are determined.

The concentrations of neomycin chosen for the two growth/death curves in Table 2 and 3 are based on the lowest concentration of neomycin known to be effective in killing non-transfected BEF cells, and two logs higher, which is the mid-range of G418 used on normal human fibroblast cells.⁴² Gancyclovir has never been used on BEF cells. Thus, the concentrations of gancyclovir chosen were based on the one half of the concentration effective for murine embryonic stem cells in targeting experiments (1 μM) and two logs-fold increase in drug. 42. Brown J. P., Wei W. and Sedivy J. M. (1997). Bypass of senescence after disruption of p. 21 cip l/wafl gene in normal diploid human fibroblasts. Science 277, 831-834.

From work in normal rat diploid cells, the level of G418 can be increased to 20 μg/ml with only 80% killing of cells transfected with a normal neo gene.⁴⁶ Thus, we expected to be able to increase the G418 concentration in BEF cells from 300 μg/ml, which is the highest amount used now, to at least 1 mg/ml using the pPNT neomycin construct. As demonstrated in rat diploid fibroblasts, cells containing the mutant neomycin gene (such as found in the pPNT vector⁴³) are more efficiently targeted.⁴⁶ Thus, a combination of high G418 with the mutant neomycin gene is optimal for efficient recovery of homologously recombined BEF cells. 43. Tybulewicz, V. L., Crawford C. E., Jackson P. K., Bronson R. T. and Mulligan R. C. (1991). Neonatal lethality and lymphopenia in mice with a homozygous disruption of the c-abl proto-oncogene. Cell 65, 1153-1163. 46. Hanson K. D. and Sedivy J. M. (1995). Analysis of biological selections for high-efficiency gene targeting. Mol. Cell. Biol. 15, 45-51.

TABLE 4
G418 and gancyclovir treatment of BEF cells transfected with pPNT.

| | G418 | GANCYCLOVIR | |
|---|---|---|---|
| VECTOR | CONCENTRATION | CONCENTRATION | PURPOSE |
| pPNT | 300 μg/ml | None | Transfected cell growth |
| | | | in G418 |
| pPNT | 1000 μg/ml | None | Transfected cell growth |
| | | | in G418 |
| pPNT | 3000 μg/ml | None | Transfected cell growth |
| | | | in G418 |
| pPNT | None | 1 μM | Transfected cell killing |
| | | | in gancyclovir |
| pPNT | None | 3 μM | Transfected cell killing |
| | | | in gancyclovir |
| pPNT | None | 10 μM | Transfected cell killing |
| | | | in gancyclovir |
| pPNT | 300 μg/ml | 1 μM | Combination experiment |
| pPNT | 1000 μg/ml | 1 μM | ″ |
| pPNT | 3000 μg/ml | 1 μM | ″ |
| pPNT | 300 μg/ml | 3 μM | ″ |
| pPNT | 1000 μg/ml | 3 μM | ″ |
| pPNT | 3000 μg/ml | 3 μM | ″ |
| pPNT | 300 μg/ml | 10 μM | ″ |
| pPNT | 1000 μg/ml | 10 μM | ″ |
| pPNT | 3000 μg/ml | 10 μM | ″ |

Southern Analysis of BEF Genomic DNA for Normal and Disrupted PRNP Gene

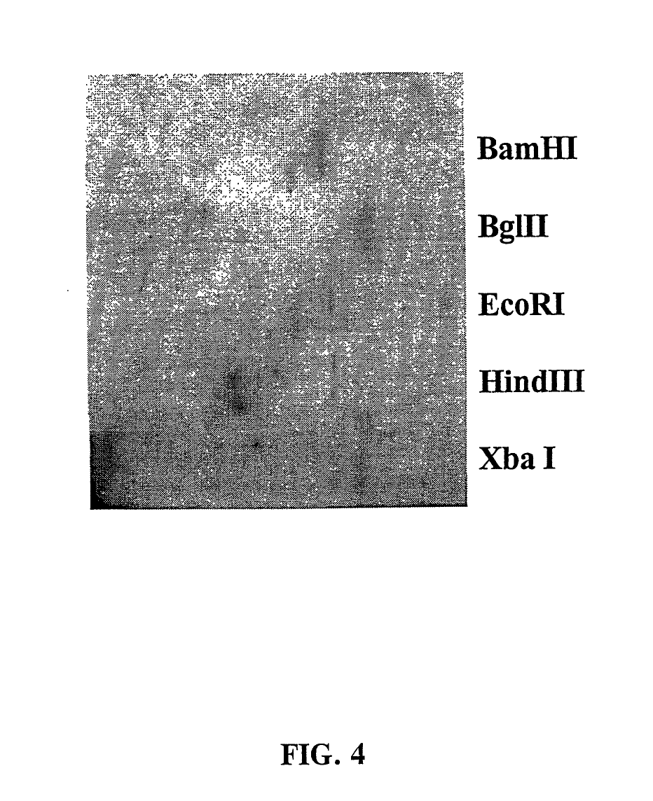

Five micrograms of normal or transfected BEF genomic DNA is digested with the selected restriction enzyme using the associated restriction enzyme buffer for 8-24 hours. The digested DNA is separated on a 1% agarose gel in standard electrophoresis buffer and transferred to a solid support membrane (nitrocellulose or nylon) using standard methods (Sambrook et al., 1989). The DNA on the membrane is hybridized to probes from the insertedtransgenes and selectable markers. Southern analysis of the normal BEF PRNP gene is shown in FIG. 4. Southern analysis of targeted BEF or other ungulate PRNP genes would reveal changes in the structure of the endogenous PRNP gene, including smaller or larger hybridizing PRNP fragments, and presence of exogenous transgenes and selectable markers.

### EXAMPLES

Bovine Fibroblast Production and Maintenance

ACT produced bovine fetal fibroblast cells (BFF) from a 55-day-old Holstein male fetus according to standard fetal fibroblast preparation. A large number of cells were prepared from this single fetus and were used to create cloned transgenic cattle. Fibroblasts are maintained in polystyrene tissue culture plates at 37° C. with 5% CO2 Cells are passed 1:10 when they reach 80% confluence. These primary cells have a 28-30 hour cell cycle and undergo approximately 30 population doublings before senescence.

Cloning of the Bovine PrP Gene

The initial plan was to obtain the prion gene in a large genomic sequence and incorporate a selectable marker in order to interrupt protein production. High molecular weight genomic DNA was extracted from bovine fetal fibroblasts. A Lambda FIX 11 Genomic Library (Stratagene) was prepared by randomly inserting restriction fragments of this genomic DNA into a phage vector and packaging it into viral particles. Free amplified product (8×10 9 plaque-forming units per ml) was used to infect E. coli and plated for isolated plaques. Blotted plaques were probed with a radio-labeled 2.4 kb Eco RI DNA fragment from plasmid pMPRP3 (ATCC) containing a DNA sequence for mouse prion. Sufficient numbers of plaques were screened in order to cover the entire genome. Phage plaques containing putative bovine PrP gene sequences were enriched, re-plated and reprobed to purify and confirm their sequence match to the mouse PrP gene. In three independent attempts at screening plaques, several initial signals were obtained and tested. None contained sequences of PrP which could be used to construct a targeting vector.

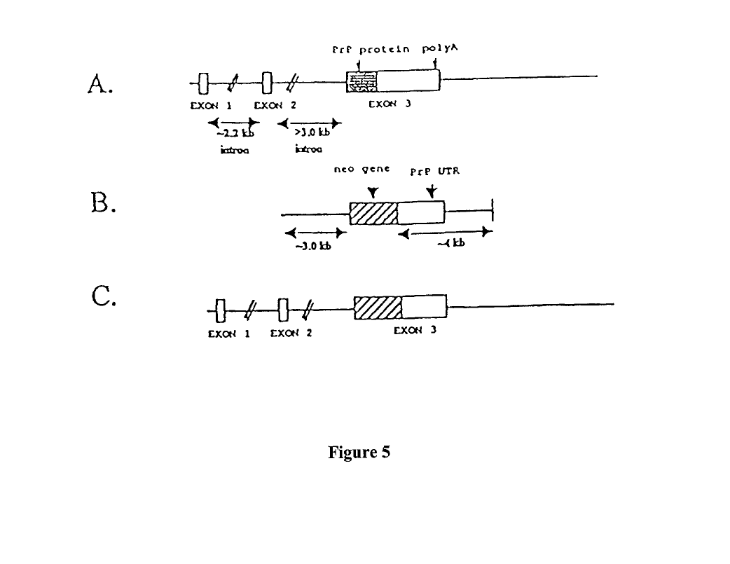

In order to obtain the genes required to build the targeting construct, PCR amplification was utilized. Primers were prepared based on sequences from GenBank AB001468 and D26150. About 2 Kb of sequence on either side of the insertion or deletion point (referred to as arms) was PCR amplified. The 5′ upstream arm of the sequence containing parts of intron I and exon 2 was amplified using the Expand PCR System (Boehringer Mannheim) by sense primer “A” (GCAGAGCT GAGCGTCTTC) and antisense primer “B” (CAGC′fCAAGTTGGATTTGTGTC). The PCR product was a 2.4 Kb DNA fragment (FIG. 5) which was cloned using a TOPO XL PCR kit (Invitrogen) and sequenced at the DNA Sequencing Facility, University of Massachusetts.

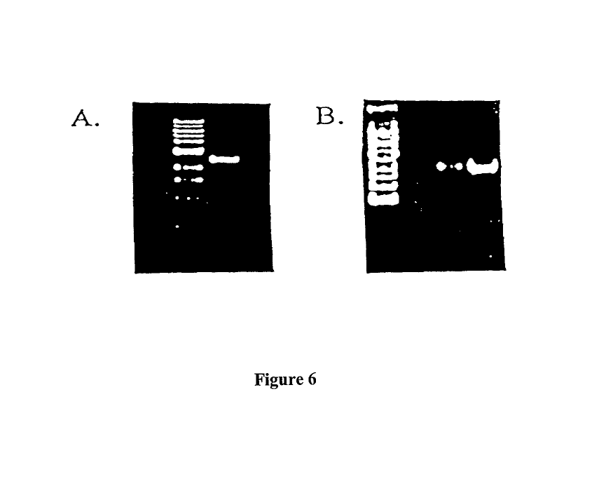

Initial work with primer C and D did not yield the desired product. An additional set of primers was needed to amplify the exon 3 sequence directly from the bovine genomic DNA. The sense primer PrP Is (GGGCAACC-I′TCCTGTTTT CATTATC) and antisense primer PrP la (CCATACACTGCACAAA-fACATTTTCGC) were used to clone a 2.129 Kb PCR product (FIG. 6.).

Targeting Vector Construction

Cloned sequences were assembled to build the targeting vector. Construction began with clone #3 of PrP 3, the plasmid that contained the coding sequence exon 3, the 3′ arm, in vector pCR-XL TOPO (Invitrogen). The cloned 5′ arm of the construct was transferred on a Sst I fragment up stream of the 3′ arm. The neo-selection (neomycin G418 resistance) cassette was modified by PCR to add a Barn HI site at the 5′ end for easier subcloning using primers TK-Bam (GCCAATATGGGATCGGCCATTGAAC) and the T7 sequencing primer (TAATACGACTCATATAGGG). This PCR product, PGK-neo, was inserted between the 3′ and 5′ arms of the on a Bam HI fragment. The final construct was linearized by Mlu I and Not I digestion, and fragments purified for transfection. When recombined with the genomic DNA this construct was intended to interrupt the sequence deleting part of exon 2, resulting in no gene product from the coding sequence in exon 3 (FIG. 5). However it failed. In retrospect there were several fundamental problems with this vector, (I) it was not promoterless neo; (2) this vector has very short left-right genomic arms contained only 2.3 kb intron 1, neo with its own promoter and 2.2 kb exon 3; (3) the 14 kb intron 2 genomic DNA was completely excluded from this vector, resulting in actually 1.5 Kb deletion.

Three constructs were used in these studies: EGFP-N1 (Clontech), pPNT and the pPRP vector that was prepared as described above. Preliminary electroporation experiments to determine the effectiveness of transfection of bovine fetal fibroblasts were done with the EGFP-NI vector (Clontech) containing a green fluorescent protein and a neomycin resistant gene. The EGFP plasmid had been successfully transfected into BFF cells in previous experiments in our lab. Use of this vector enabled easy detection of transfected cells by examination under fluorescent microscopy. Transfected BFF cells and resistant colonies fluoresced green under ultraviolet light. The use of this EGFP vector in the testing of electroporation conditions for BFF cells is indicated in Table I. Electroporation parameters were modified for the second transfection with EGFP and the subsequent transfection with pPNT vector. Successful transfection with BFF cells had been done routinely at 400 volts and 250 uF capacitance in our lab. In a similar experiment done by K. D. Wells et al. (abstract at LETS meeting 1998), BFF cells were transfected at 0, 200, 300, 400 or 500 volts with a capacitance of 500 uF to induce DNA uptake. Maximum transfection was obtained at 400 and 500 volts. Electroporation parameters were focused between 450 and 650 volts, with 400 volts being considered the baseline voltage. Higher voltages were tested, by increasing voltage in increments of 50 volts.

Drug selection was tested by growing transfected and untransfected bovine fetal fibroblasts under various concentrations of geneticin (G418)--400 to 3000 ug/ml) In our study, 400 ug/ml G418 for a period of ten days was the optimal drug selection for bovine fetal fibroblasts, producing stable, neomycin-resistant colonies.

Electroporation

Bovine fetal fibroblasts were grown to 80% confluence in DMEM-high glucose media (Gibco/BRL) with 15% FBS (Hyclone). The cells were harvested with 1X Trypsin/ EDTA (Gibco/BRL) and then centrifuged at 1200 rpm for 7 minutes at room temperature to form pellets. Cells were washed and resuspended in Ca+2/Mg+2 free Dulbecco's PBS at a density of 5×106 cells/ 0.5 ml. For each electroporation experiment, a 500 pi aliquot of resuspended cells and 20 Erg of linearized DNA in 25 ul sterile water is transferred to an electroporation cuvette (Biorad) with a 0.4 cm gap width. The cells and DNA are mixed by gently tapping the cuvette and then incubated on ice for ten minutes. After incubation, the cells and DNA are again mixed gently and then electroporated in an Invitrogen II Electroporator with the parameters in Table 5 below:

TABLE 5
Optimization of electroporation conditions for BFF cells

| | | |
|---|---|---|
| DNA | VOLTAGE | CAPACITANCE |

| | | | |
|---|---|---|---|
| None | 0 v | 0 | uF |
| None | 100 v | 500 | uF |
| None | 300 v | 500 | I& |
| None | 300 v | 250 | uF |
| None | 450 v | 250 | p F |
| None | 600 v | 250 | uF |
| None | 600 v | 71 | uF |
| None | 800 v | 71 | uF |
| EGFP | 0 v | 0 | uF |
| EGFP | 100 v | 500 | uF |
| EGFP | 300 v | 500 | uF |
| EGFP | 300 v | 250 | uF |
| EGFP | 450 v | 250 | uF |
| EGFP | 600 v | 250 | uF |
| EGFP | 600 v | 71 | uF |
| EGFP | 800 v | 71 | uF |

The cuvettes are placed back on ice for an additional 10 minutes following electroporation. Prior to plating the electroporated cells, 100 ul of Ca 2/Mg+2 free DPBS is added to each cuvette and the cells are gently mixed. Under sterile conditions, 10 ml of DMEM-high glucose with 15% FBS is added to each of six 20×100 mm 2 polystyrene tissue culture dishes. A 100 ul aliquot of electroporated cells is removed from the cuvette and plated onto each of six tissue culture dishes. All six plates of cells are grown overnight in drug-free media at 37° C. with 5% CO2 atmosphere. The next morning three plates of transfected cells were harvested by trypsinization and cell counts were done to determine cell survival. Drug selection was begun on the remaining three plates by changing the media and adding 400ug/ml geneticin (G418) to each plate. The cells were then grown at 37° C., 5% CO2 under G418 selection for 10 days. The media in these plates was changed daily to maintain a constant level of G418 for drug selection. After 10 days of G418 selection, visible colonies were present and the number of colonies on each plate was counted. The average colony count from the three plates was used to determine transfection efficiency for each electroporation condition. Each electroporation condition was tested in two separate experiments.

Prior to transfection of bovine fetal fibroblasts with the pPNT vector, preliminary electroporation experiments were done with the EGFP-N 1 vector (Clontech) containing a green fluorescent protein and a neomycin resistant gene. The EGFP plasmid had been successfully transfected into BFF cells in previous experiments in our lab. Use of this vector enabled easy detection of transfected cells by examination under fluorescent microscopy. Transfected BFF cells and resistant colonies fluoresced green under ultraviolet light. The use of this EGFP vector in the testing of electroporation conditions for BFF cells is indicated in Table 1. Electroporation parameters were modified for the second transfection with EGFP and the subsequent transfection with pPNT vector. Successful transfection with BFF cells had been done routinely at 400 volts and 250 uF capacitance in our lab. In a similar experiment done by K. D. Wells et al., BFF cells were transfected at 0, 200, 300, 400 or 500 volts with a capacitance of 500 uF to induce DNA uptake. Maximum transfection was obtained at 400 and 500 volts.

Electroporation parameters were focused between 450 and 650 volts, with 400 volts being considered the baseline voltage. Higher voltages were tested, by increasing voltage in increments of 50 volts.

TABLE 6
Electroporation parameters for BFF cells with EGFP and pPNT.

| DNA | VOLTAGE | CAPACITANCE |
|---|---|---|
| EGFP | 0 v | 0 uF |
| EGFP | 450 v | 250 uF |
| EGFP | 550 v | 250 uF |
| EGFP | 600 v | 250 uF |
| EGFP | 650 v | 250 uF |
| PPNT | 450 v | 250 uF |
| PPNT | 550 v | 250 uF |
| PPNT | 600 v | 250 uF |
| PPNT | 650 v | 250 uF |

A second group of transfections of BFF cells with pPNT was done using the electroporation parameters described in Table 6. These same electroporation conditions were used for the transfection of the pPRP target vector.

Test selection of untransfected BFF cells.

Untransfected bovine fetal fibroblasts were plated at a density of 5×10⁶ cells in 10 ml of DMEM-high glucose media with 15% FBS onto 20×100 mm 2 polystyrene tissue culture dishes. The cells were grown overnight at 37° C. with 5% CO,. The media was changed and drug selection with geneticin (G418) was begun the next morning

Table 7 contains the drug concentrations that were tested. G418 selection was done for 10 days, by which time there was complete killing of the untransfected cells. Two plates of BFF were used for each drug concentration and cell counts were done on these plates of cells at 0, 3, 7 and 10 days. Previous work in our laboratory had determined that 400 ug/ml) neomycin was sufficient to kill non-neomycin containing BFF cells, but would allow the rapid proliferation of BFF cells containing a transfected neomycin gene. Thus, for this experiment drug selection was begun at 400 ug/ml) geneticin (G418) and increased drug concentrations of 600, 800 and 1000 ug were tested.

TABLE 7
Geneticin (G418) treatment of untransfected BFF cells

| | 6418 | |
|---|---|---|
| DNA | Concentration | Purpose |
| None | 0 ug | Control |
| None | 400 ug | Normal cell killing by |
| | | 6418 |
| None | 1000 ug | Normal cell killing by |
| | | 6418 |
| None | 3000 ug | Normal cell killing by |
| | | 6418 |

A second mortality curve was done with untransfected bovine fetal fibroblasts with drug selection begun at 400 ug/ml) G418 and increased to concentrations of 600, 800 and 1000 ug for testing. Table 8 contains the drug concentrations that were tested.

TABLE 8
Geneticin (G418) treatment of untransfected BFF cells

| | 6418 | | |
|---|---|---|---|
| DNA | Concentration | | Purpose |
| None | 400 | ug | Normal cell killing by |
| | | | 6418 |
| None | 600 | ug | Normal cell killing by |
| | | | 6418 |
| None | 800 | ug | Normal cell killing by |
| | | | 6418 |
| None | 1000 | pig | Normal cell killing by |
| | | | 6418 |

Test Selection of Transfected BFF Cells

BFF cells were transfected with pPNT vector containing both a neomycin resistant gene as well as a thymidine kinase gene. The cells were electroporated at 450 volts and a capacitance of 250 uF to induce DNA uptake. Following electroporation, cells cloned transgenic calves produced from non-quiescent fetal fibroblasts were plated at a density of 5×106 cells in 10 ml media/ 100 mm 2 plate and incubated overnight at 37° C. with 5% CO2 atmosphere. The media was changed and drug selection with G418 was begun the next morning. The drug concentrations we tested are listed in Table 8. Geneticin selection was continued for 12 days by which time resistant colonies were visible. Two plates of BFF were used for each drug concentration and cell counts were done on these plates of cells at 0, 4, 7 and 12 days. As previously noted, drug selection was begun at 400pg/ml G418 and increased geneticin concentrations were tested. The concentrations of neomycin chosen for the two growth/kill curves in Tables 7 and 9 are based on the lowest concentration of neomycin known to be effective in killing non-transfected BEF cells, and two logs higher, which is the mid-range of G418 used on normal human fibroblast cells).

TABLE 9
Geneticin (G418) treatment of BFF cells transfected with pPNT

| | 6418 | |
|---|---|---|
| DNA | Concentration | Purpose |
| PPNT | 0 ug | Transfected cell |
| | | growth |
| | | in G418 |
| PPNT | 400 ug | Transfected cell |
| | | growth |
| | | in G418 |
| PPNT | 1000 ug | Transfected cell |
| | | growth |
| | | in G418 |
| PPNT | 3000 ug | Transfected cell |
| | | growth |
| | | in G418 |

Results of Electroporations

No spontaneously resistant colonies occurred in the electroporation of untransfected bovine fetal fibroblasts in which no DNA was present. Cell survival decreased sigmoidally with the increasing voltages tested. Cells were electroporated at 0, 100, 300, 450, 600 and 800 volts with capacitance ranging from 0 uF to 500 uF At 100 volts, there was 89% cell survival and at 800 volts, cell survival had decreased dramatically to 1.2%. Table 6 indicates the total and average cell counts for three plates following 10 days of drug selection with 400 ug/ml) geneticin (G418). Transfection efficiency could not be calculated for this experiment in the absence of resistant colonies.

TABLE 10
Electroporation of untransfected BFF cells

| | | Total | Average # | % | Average # |
|---|---|---|---|---|---|
| Voltage | Capacitance | cells | cells/plate | survival | colonies/plt. |
| 0 v | 0 uF | 1.84 × 10 | 6.10 × 10 | 100.0 | 0 |
| 100 v | 500 uF | 1.63 × 10 | 5.43 × 10 | 89.0 | 0 |
| 300 v | 500 uF | 0.58 × 10 | 1.92 × 10 | 31.5 | 0 |
| 300 v | 250 uF | 1.03 × 10 | 3.43 × 10 | 56.2 | 0 |
| 450 v | 250 uF | 0.26 × 10 | 0.87 × 10 | 14.2 | 0 |
| 600 v | 250 uF | 0.10 × 10 | 0.34 × 10 | 5.7 | 0 |
| 600 v | 71 uF | 0.11 × 10 | 0.35 × 10 | 5.8 | 0 |
| 800 v | 71 uF | 0.01 × 10 | 0.07 × 10 | 1.2 | 0 |

Electroporation of transfected BFF cells was done as a series of experiments using several DNA constructs EGFP-N I (Clontech), pPNT and pPRP. The EGFP construct was used in the first electroporation experiment as it had been successsfully transfected into BFF cells previously in our lab. This construct contains a neomycin resistant gene and a green fluorescent protein, enabling easy detection of transfected BFF cells under fluorescent microscopy. Transfected BFF cells were fluorescent green under ultraviolet light. Cells were transfected at 0, 100, 300, 450, 600 and 800 volts with a capacitance range of 0 to 500 uF As seen previously in the untransfected BFF cells, cell survival decreased with increasing electroporation voltages. Total cell and average cell counts for three plates following 10 days of drug selection are shown in Table 11.

TABLE 11
Electroporation of BFF cells transfected with EGFP

| | | | Average # | % | Average # |
|---|---|---|---|---|---|
| Voltage | Capacitance | Total Cells | cells/plate | survival | colonies/plt. |
| 0 v | 0 uF | 1.64 × 10° | 5.46 × 10 | 100 | 0 |
| 100 v | 500 uF | 1.64 × 10 | 5.46 × 10 | 100 | 0 |
| 300 v | 500 uF | 0.74 × 10° | 2.45 × | 44.9 | 7 |
| | | | 105 | | |
| 300 v | 250 uF | 0.59 × 10 | 1.97 × 10 | 36.1 | 4 |
| 450 v | 250 uF | 1.04 × 10 | 3.46 × 10 | 63.4 | 2 |
| 600 v | 250 uF | 0.15 × 10 | 0.49 × 10 | 8.9 | 14 |
| 600 v | 71 uF | 0.70 × 106 | 2.33 × 10 | 42.7 | 1 |
| 800 v I | 71 uF | 0.41 × 10 | 1.36 × 10 | 24.9 | 8 |

Maximum transfection occurred at 600 volts, 250 uF with an average of 14 individual resistant colonies present on each plate. Cell survival was only 8.9% at this voltage, yet these cells yielded the highest number of colonies per plate. Similar results were reported in an electroporation experiment. As described by others, with increasing voltages, cell survival decreased in a sigmoidal fashion and conversely, the number of surviving cells that were transfected increased sigmoidally with increased voltage[40]. Duplicate transfections were done simultaneously in the next electroporation experiment. The EGFP and the pPNT constructs were transfected into BFF cells. A more focused range of electroporation conditions were used for these transfections based on the maximum transfection (600 volts) obtained in our previous experiment. Successful transfections were routinely done at 400 volts and K. D. Wells et al. obtained maximum transfection at 400 and 500 volts in BFF cells [40]. Therefore, cells were transfected at 0, 450, 550, 600 and 650 volts to achieve optimal transfection efficiencies. Maximum transfection occurred at 600 volts, 250 uF in the BFF transfected with EGFP construct; an average of 15 resistant colonies per plate. Similar results were obtained in the pPNT transfection, the highest yield of resistant colonies (20/plate) occurred at 600 volts and 250 uF capacitance. As previously noted, cell survival decreased and transfection efficiency increased with increased electroporation voltages.

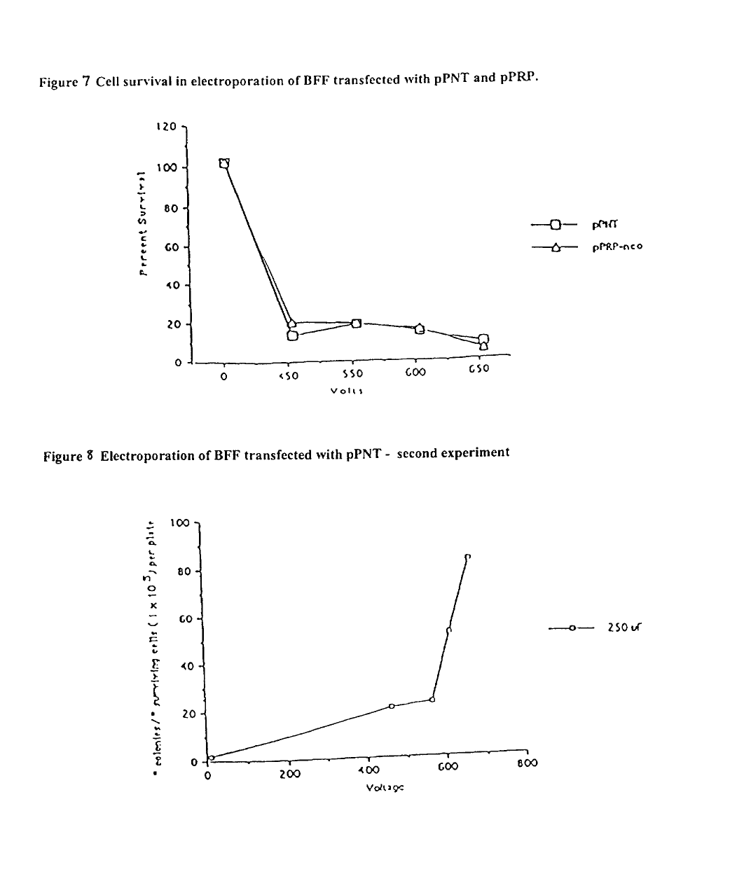

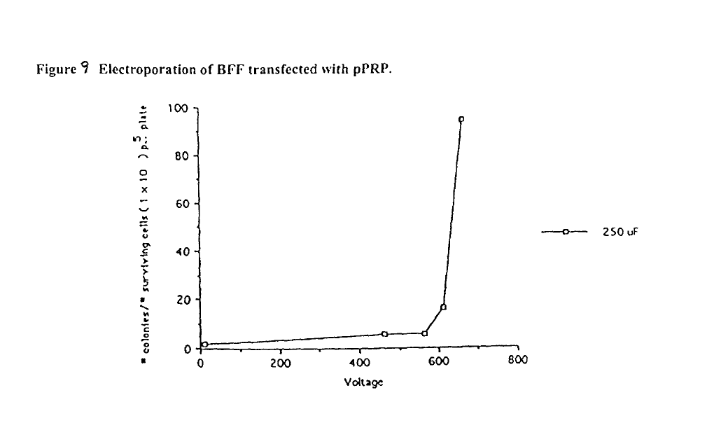

The voltage range of 450 to 650 volts with 250 VF capacitance was used for the subsequent electroporation experiments. Once again two separate transfections were done simultaneously, a second transfection of BFF cells with pPNT and the first attempt with the target construct pPRP. Maximum transfection was obtained at 600 volts and 250 uF capacitance for the pPNT vector. An average of 28 resistant colonies per plate was achieved for the pPNT transfection. For the target vector, pPRP, maximum transfection was obtained at a slightly higher voltage, 650 volts with 250 uF capacitance. Twelve resistant colonies per plate were recorded for the pPRP transfection. At these higher electroporation voltages, the sigmoidal pattern of decreasing cell survival and increasing transfection efficiencies was evident. These results are contained in FIGS. 7-9.

Transfection of BFF cells with pPRP was repeated in a second experiment using the same voltages and capacitance as stated above. Once again, maximum transfection was obtained at 600 volts and 250 uF The number of resistant colonies obtained in this repeat transfection was comparable to those obtained in the previous pPRP experiment.

Optimization of Drug Selection

Untransfected bovine fetal fibroblasts were grown under various concentrations of geneticin (G418). A concentration of 400 ug/ml) G418 was routinely used for transfection experiments done by our lab, and this drug concentration was found to be sufficient to kill non-neomycin containing BFF cells, but would allow rapid growth of BFF cells transfected with a neomycin gene. Therefore, 400 ug/ml) G418 concentration was considered the starting point for this experiment and increased drug concentrations were tested. In the first experiment a broad range of drug concentrations; 400, 1000 and 3000 ug/ml) G418 were tested (Table 12). At 400 ug/ml) G418, the untransfected BFF cells continued to grow vigorously for three days following the onset of drug selection. Untransfected BFF cells grew at a reduced rate for this same time period under 1000 pg/ml G418. Within 3 days of drug selection at 3000 ug/ml) G418, the BFF cells were dead (FIG. 11). Ten days of drug selection with 400 ug/ml) G418 was required to kill the untransfected BFF cells. Two plates of cells were used for each drug concentration and cell counts were done at each time interval. Table 12 contains the average cell counts for each G418 concentration tested at the various time periods.

TABLE 12
G418 Drug selection of untransfected I3hF cells

| Days of | Cell count | Cell count | Cell count |
|---|---|---|---|
| selection | 400 Vg G418 | 1000 ug G418 | 3000 ug G418 |
| 0 | 0.57 × 10 | 0.57 × 10″ | 0.57 × 10 |
| 3 | 2.52 × 10 | 1.70 × 106 | 0.10 × 10 |
| 7 | 2.78 × 10 | 0 | 0 |
| 10 | 0.04 × 10 | 0 | |

The range of drug concentrations was focused between 400 and 1000 ug/ml) G418 for the second kill curve with untransfected BFF cells. There was a reduced rate of cell proliferation at the higher drug concentrations (600, 800 and 1000 ug/ml) for the first few days after drug selection was begun. After seven days of G418 treatment, total mortality of the BFF cells had occurred with 800 ug G418 and only a small number of untransfected cells were surviving with 600 ug G418. It is apparent from both of these kill curves that there was a lag time of approximately 3 days in drug selection. During this time, BFF cell growth continued even at reduced rates with increased G418 concentrations.

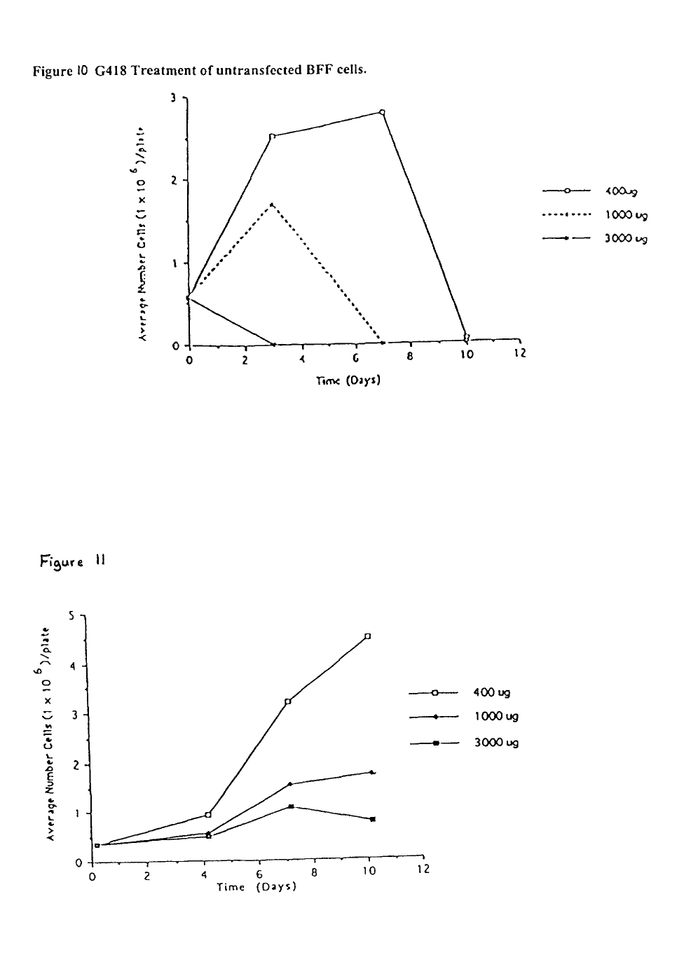

Bovine fetal fibroblasts were transfected with the pPNT construct and under went drug selection for twelve days. A broad range of drug concentrations were tested; 400, 1000 and 3000 ug/ml) G418. No mortality occurred after 12 days of drug selection for any of the concentrations tested. BFF cells continued to grow at reduced rates for these higher drug concentrations (FIGS. 10 and 11). Table 13 contains the average cell counts for two plates of BFF taken over a 12 day period. Due to difficulties in obtaining gancyclovir, no test selection of BFF cells was conducted with this drug.

TABLE 13
G418 Treatment of BFF cells transfected with pPNT

| Days of | Cell count | Cell count | Cell count |
|---|---|---|---|
| selection | 400 ug G418 | 1000 ug G418 | 3000 ug G418 |
| 0 | 0.26 × 10⁶ | 0.26 × 10 | 0.26 × 10 |
| 4 | 0.84 × 10 | 0.49 × 10 | 0.42 × 10 |
| 7 | 3.10 × 10 | 1.45 × 10 | 0.99 × 10 |
| 12 | 4.38 × 10 | 1.63 × 10 | 0.67 × 10 |

Conclusion

The objectives of this experiment were to clone genomic sequences of bovine priors gene (PrP), to create a targeting vector, and optimize the conditions for electroporation and drug selection in bovine fetal fibroblast cells. The targeting vector that was created was not successful, apparently because (1) it was not promoterless neo; (2) this vector has very short left-right genomic arms contained only 2.3 kb intron 1, neo with its own promoter and 2.2 kb exon; and/or (3) the 14 kb intron 2 genomic DNA was completely excluded from this vector, resulting in actually 15 kb deletion. Accordingly, an alternative strategy for the construction of the targeting vector was developed that should solve these problems that is detailed in Example 2.

### Example 2

Generation of a DNA Probe for- Isolation of Pr-P Gene From a Bovine Genomic DNA Library.

PCR primers (5′ primer, ATGGTGAAAAGCCACATAG; 3′ primer, TATCCTACTATGAGAAAAAT) are designed so that the DNA sequences of the PCR product correspond to the PrP open reading frame which is part of the PrP exon 3 . The predicted size of the PCR product is 794 bp.

Screening Genomic DNA Library and Identification of PrP Genomic DNA.

A bovine genomic DNA library, which has been built, will be screened with the 794 by PrP probe labeled with nonisotopic digoxigenin-dUTP (Roche Molecular Biochemicals). We have successfully cloned two genomic DNAs with such a labelling system. The identified PrP genomic DNA will be confirmed with partial DNA sequencing, and mapped for subsequently construction of gene targeting vectors.

Construction of Gene Targeting Vector.

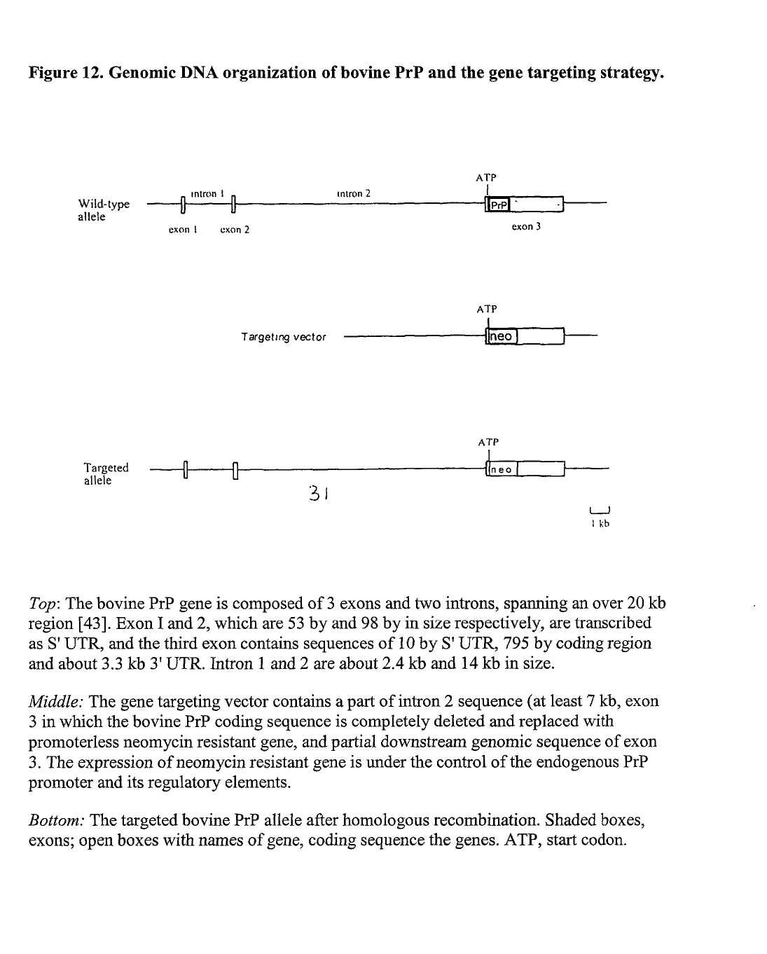

An about 10 kb PrP genomic DNA is needed as left and right arms of targeting DNA fragment for homologous recombination. The complete PrP coding sequence (795 bp) is deleted from the Exon 3, and replaced with promoterless neomycin resistant gene. We hope to isolate bovine PrP genomic DNA fragment which is the region shown in FIG. 12.

Design a probe for genotyping PrP targeted BEF and animals.

Once a PrP genomic DNA fragment was isolated, mapped and demonstrated to meet the requirements for the construction of the targeting vector, a 0.5 to 1.0 kb PrP genomic DNA, excluded from targeting vector, will be determined as a probe for genotyping gene targeted alleles. This probe is labeled with non-isotopic digoxigenin-dUTP (Roche Molecular Biochemicals), and tested in a Southern blot analysis for partially digested genomic DNA from wild-type. We have successfully performed Southern blot analysis with such labeling method

If necessary, multiple rounds of screening of the genomic DNA library will be effected, as it is possible that screening bovine genomic DNA library several times may be required in order to isolate DNA fragments which cover genomic regions necessary for building a targeting vector.

Use of the Promoterless Targeting Vector (Aim I) to Carry out Homologous Recombination in Bovine Fetal Fibroblasts to Identify Gene-Targeted Cells with a Null-Mutation on one Allele of the PrP Gene.

Bovine embryonic fibroblast (BEF) will be produced from a 35 to 40-day-old Holstein male fetus by ACT according to standard fetal fibroblast preparation methods. A large number of cells from this single fetus will be prepared. Analagously prepared cells have been successfully used in the past to create cloned transgenic cattle. Fibroblasts are maintained in polystyrene tissue culture plates at 37° C., 5% CO2 Cells are passed 1:10 when they reach 80% confluency. These primary cells have a 28-30 hour cell cycle and undergo approximately 30 population doublings before senescence.

Introduction of PrP Gene Targeting Constructs into BFF.

A total of 1×10′ BEF (80% confluency) are harvested by trypsinization, and resuspended at a density of 5×10⁶ cells/450 ul of ice cold PBS. A duplicate of electroporation is performed. For each electroporation, a 2S-SO ug of DNA targeting constructs in SO ul of PBS is mixed with 450 ul resuspended BFF in an electroelution cuvette, and incubated on ice for 10 minutes. Following the ten minute incubation the cells are again gently resuspended and then electroporated with the parameters of 600 volts and 250 uF (Invitrogen II Electroporator). Following the pulse, the cuvettes are again incubated on ice for an additional 10 minutes. The electroporated BFF are transferred and resuspended in 10 ml of the above media, and plated onto ten 100 mm 2 polystyrene tissue culture dishes with 5×10⁵ of cells per dish. The cells are incubated at 37° C., 5% CO2 incubator.

Culture of PrP Gene Targeted BFF in Selection Medium Containing G418.

Selection medium containing 400 ug/ml G418 will be added to transfected BEF after 48-hour culture in normal medium following electroporation. The transfected BEF will be maintained in selection medium for about 10 days or till surviving colonies form. The concentration of G418 will be adjusted accordingly in order to select either heterozygous cells or possibly homozygous cells if higher concentration of G418 is supplemented to culture medium.

Expansion and Genotyping of Surviving Colonies of BEF After Selection.

Surviving colonies of BEF will be isolated individually with cloning rings, and expanded in selection medium. A duplicate culture for each surviving colony is needed. One set is for extraction of genomic DNA required for genotyping, and the other set is for freezing as stocks. Genotyping of surviving colonies for gene targeting will be obtained with PCR approach as a primary screening followed by Southern blot analysis.

Generation of a PrP Heterozygous Knock Out (KO) Bovine Fetuses by Nuclear Transfer Using Gene-Targeted Cells

Gene targeting is a technique that requires cell selection with antibiotics in order to isolate targeted-cell-colonies which derived from a single cell through multiple doublings. Since we are proposing to work with primary cell lines, by the time a clonal cell line emerges there are barely any population doublings left for cell expansion. In 1998 and 2000, we published work that has demonstrated the capacity of somatic cell nuclear transfer to completely rejuvenate cell lines. This characteristic will allow us to perform homozygous gene targeting, the first one using primary cells from a naturally produced fetus and the second one using primary cells from a cloned fetus.

Previous work on bovine somatic cell nuclear transfer has demonstrated that this technique is repeatable not only with primary cells from fetuses and adult animals but with transgenic cells as well. In our laboratory we are capable of producing transgenic animals by cloning at a reasonably high efficiency. Forty to fifty percent of the recipient cows (two blastocysts per cow) became pregnant. This overall efficiency however does not reflect the variation between cell lines. We have observed that not all clonal lines, although originated from the same genome, will maintain the same level of efficiency (measured by generation of healthy fetuses).

We will then generate male fetuses using 10 different cell lines. Line 1 will be non-transgenic fetal fibroblasts from the same genome as the targeted cell line. Lines 2 through 10 will be PrP homozygous KO cells. Efficiency to develop to blastocyst stage will be measured as well as pregnancy rates at 30 to 35 days of gestation and capacity to generate healthy 40 day old fetuses.

The number of embryos (blastocysts) to be produced per cell line will be SO to be transferred into 25 cows. Since the magnitude of this work will not allow us to perform the whole experiment in one day, we will divide each cell line into 3 different replicates (one a day) and randomize all the different treatments.

| | | | | | | |
|---|---|---|---|---|---|---|
| | No. of | | | No. of | No. of cows | No. of |
| | reconstructed | No. of | % of | blastocysts | pregnant at | healthy |
| Cell line | embryos | blastocysts | blastocysts | transferred | 30-35 days | fetuses |

```
  1 control
  2
  3
  4
  S
  6
  7
  8
  9
10
```

Expected results: Based upon our previous work using transgenic cells, we would expect to have cell lines that are incapable of generating pregnancies as well as cell lines that can generate pregnancies at a rate of 40 to 50% with a 80 to 90% production of healthy fetuses. If more than one cell line produces healthy fetuses, we will choose five that have the best efficiency to generate the second round of gene targeting.

However, it is possible that the pregnancy rate is below average for all cell lines. In this case we will prescreen cell lines from different genotypes including different breeds.

Egg Retrieval and Maturation

Ovaries will be recovered at a slaughterhouse, placed in wane PBS (34° C.) and brought to the laboratory within a limit of 8 hours. Each follicle of more than 2 mm in diameter will be aseptically aspirated with an 18 G needle. Search of oocytes will be performed in modified Tyrode's medium (TL Hepes). Oocytes with a homogeneous cytoplasm, considerable periviteline space and intact cumulus cells will be placed in maturation medium M 199 (GIBCO), 10% FCS, S ul/ml bFSH (Nobly, 5 ul/ml bLH (Nobly and 10 ul/ml Pen-strep (Sigma) for 22 h at 38.5 C and 5% CO2 It is expected that 70 to 80% of the eggs placed in maturation will be capable to reach metaphase II stage.

Donor-Cell Preparation

Cell lines will be isolated from a 35 to 40 days old bovine fetus as follows. Under sterile conditions, liver intestines and head of the fetuses will be discarded. The remained part of the fetus will be carefully minced and placed in a solution of DPBS with 0.08% trypsin (Difco) and 0.02% EDTA (Sigma). After 30 min incubation at 37° C. the supernatant will be discarded and the pellet resuspended with Trypsin-EDTA/DPBS. After 30 minutes incubation, the supernatant will be removed and centrifuged at 300 g for 10 minutes. Pellet will be resuspended in culture media (DMEM+15% FCS, 4 ul/mI Antibiotic-antimycotic, 2.8 ul/mI 2-Mercaptoethanol, 0.3 mg/ml L-glutamine) and plated in Polystyrene tissue culture dishes (Corning 25010). After 2 passages mostly fibroblast-like cells will be in the culture. These cells will be used as control or for further gene targeting experiments.

Egg Enucleation

Eighteen hours post maturation; metaphase II oocytes will be placed in a 100-ul drop of TL HECM-Hepes under mineral oil (Sigma). Oocyte enucleation (extraction of chromosomes) will be performed using a beveled glass pipette of 25 um diameter. Evaluation of enucleation will be done by exposure of individual oocytes previously cultured for 15 min. in 1 ug/ml) of bisBENZIMIDE (Hoechst 33342, Sigma) in TL HECM-Hepes under UV light.

Cell Transfer

Donor cells will be selected at G 1 (proliferating) stage using the shake-off method described elsewhere. Briefly, cells are cultured at SO to 60% confluency in the presence of culture media with 15% FCS. A few minutes prior to the cell transfer procedure, the plate is vortexed for 30 to 60 seconds at speed 3. Media is later collected and centrifuged at 300 g for 10 minutes. The pellet is then resuspended in Hecm Hepes media and cell used for nuclear transfer. Using a 20 microns internal diameter glass pipette, one cell will be loaded and placed in the periviteline space of the egg.

Cell fusion

The enucleated egg and the donor cell will be fused with the egg's cytoplasm at 23 hours post maturation according with conditions previously described. Briefly, enucleated eggs will be electrically fused with the donor cell using an electrical pulse of 2.5 kV-cm for 10 to 1 S microseconds in 0.3 M manitol (Sigma).

Nucleus Transfer- Unit (NTU) Activation

Fused embryos now called NTU will be activated chemically 2 hrs after cell fusion using chemical activation protocol consisting of placing NTU in media containing 10 micromoles of Ionomicyn followed by a 8 hours incubation in cycloheximide and cytochalasin-B

Embryo Culture

After activation and during the first 72 hrs after activation, embryos will be cultured in 500 ul well plates with mouse embryonic fibroblast (MF) feeder layers and ACM media with 6 mg/ml BSA. On day 4, embryos were transferred to 500 ul well plates with mouse fibroblasts (MF) feeder layers, ACM media 6 mg/ml BSA and 10% FCS until blastocyst stage (day 7 or day R after activation)

Embryo Transfer and Pregnancy Check

Embryo transfer will be performed as described elsewhere. Briefly, two blastocysts grade 7-I or 7-2 (IETS classification) will be non-surgically placed in the uterine horn, ipsilateral to the corpus luteum, 6 to 7 days after the onset of estrous. Access to the horns will be via trans-cervical catheters. Pregnancy check will be performed by rectal ultrasound at 35 days post embryo transfer. Presence of heartbeat will indicate a healthy pregnancy.

Fetus Retrieval

Once heartbeat is obtained, fetuses will be retrieved at 40 days after embryo transfer via laparotomy. The fetus/ fetuses will be removed from the uterus and without removing the placental sac placed in a SO ml tube with PBS and antibiotic send to the laboratory at 4 degrees C.

Calves Delivery

Three weeks prior to due date (280-285 days) recipient cows will be brought to the barn and monitored 24 hrs for any sign of early parturition. A week prior to delivery and 24 hrs prior to the C section, an IM injection of dexametasone will be administered to the recipient cow in order to trigger maturation of the calf s lungs. The next day, a C-section will be performed. Upon birth, the calf will be administered surfactant and monitored constantly until all his vital signs are stable. Pasteurized calostrum will be made available and administered to the calf upon first suckling reflex is observed.

Genotype Cloned Fetuses and Isolate PrP Heterozygous KO Fetal Fibroblasts.

Genotyping Cloned Fetuses

Genomic DNA is extracted from tissues of the cloned bovine fetuses, and genotyped with Southern blot analysis with the same probe as for genotyping the gene targeted BEF which are used for generating the cloned fetuses.

Isolation of low Passages Fetal, Fibroblasts with PrP Heterozyous Knockout.

Primary culture of BFF permit only a limited number of cell population doublings. Several of these doubling times will be utilized during the procedures of homologous recombination in BEF. At the point at which BFF with PrP heterozygous knockout are identified, they may possibly be near senescence and not adequate for one more round of homologous recombination. We anticipate that it will be necessary to isolate new BFF from cloned fetuses with PrP heterozygous knockout, and these new BFF will be used for a second-round of gene targeting to obtain PrP homozygous knockout BFF as described previously.

The method to isolate and maintain PrP heterozygous knockout fetal fibroblasts is essentially the same as described previously, except that PrP heterozygous knockout fetuses will be the origin of the cells.

Homologous Recombination in PrP Heterozygous KO Fetal Fibroblasts and Identify Gene- Targeted Cells with Null-Mutations on both Alleles of the PrP Gene

The method is essentially the same as the one described previously, except (1) PrP heterozygous knockout BFF are used for this second-round of gene targeting and (2) G418 concentration in selection medium is optimized to favor the survival of cells with PrP homozygous knockout. In a normal situation, the G418 concentration is needed to be double of that for selecting PrP heterozygous knockout cells, i.e. 800 ug/ml in culture medium.

Pitfalls and Anticipated Difficulties

Fewer colonies than needed will survive selection medium with high concentration of G418. To ensure enough colonies being obtained after selection, triple or quadruple sets of electroporation will be carried out. Based on our own experiences in other knockouts, there is a possibility that we could have a difficult time to obtain PrP homozygous knockout cells in the second round of gene targeting because of very low efficency if the same targeting vector is used. The reason is speculated to be unknown that (1) the same targeting vector, which has no mismatch with the targeted allele of PrP heterozygous knockout cells, may tend to recombine with the targeted allele; (2) the use of the same selection marker will not discriminate heterozygous and homozygous knockout cells. To overcome this potential problem should it arise, we will build a gene targeting vector containing a different selection marker, i.e. hygromycin, for the second round of gene targeting.

Generate PrP Homozygous KO Bovine Calves by Nuclear Transfer Using PrP Homozygous KO Cells

In principle, we will repeat the experimental design as described previously, but in this case with 6 cell lines, one control and S targeted. Efficiency to develop to blastocyst stage will be measured as well as pregnancy rates at 30 to 35 days of gestation and capacity to generate healthy newborn calves. The number of embryos (blastocysts) to be produced per cell line will be 50 to be transferred into 25 cows. Since the magnitude of this work will not allow us to perform the whole experiment in one day, we will divide each cell line into 3 different replicates (one a day) and randomize all the different treatments.

| | | | | | | |
|---|---|---|---|---|---|---|
| | No. of | | | No. of | No. of cows | No. |
| Cell | reconstructed | No. of | % of | blastocysts | pregnant at | of healthy |
| line | Embryos | Blastocysts | blastocysts | transferred | 30-35 days | calves |

1 control
2
3
4
5

Expected results: When two embryos are transferred per recipient cow, the overall pregnancy rate does not differ from embryos produced by conventional artificial insemination followed by embryos transfer. For reasons not yet understood however, there is significant increase of abortions in clone fetuses. Between 50 to 90 days of gestation and 220 to 280, half of the cows diagnosed pregnant will abort. In our study with KO cell lines we expect the efficiency to generate healthy calves to vary considerably between lines. The overall efficiency should be between 10 to I S % (cows transferred/healthy calves born).

### REFERENCES

1. Scott et al (1999), Proc. Nat'l. Acad. Sci. USA, 96:15137-15142.

2. Schmerling et al. (1998), Cell 93:203-214.

3. Horiuchi et al (1998), Animal Genetics, 29:37-40.

4. Cibelli J. B., Stice S. L., Golueke P. J., Kane J. J., Jerry D. J., Blackwell C., Ponce de Leon F. A. and Robl J. M. (1998). Cloned transgenic calves produced from non-quiescent fetal fibroblasts. Science, accepted.

5. (1995). Transmissible Spongiform Encephalopathies: A summary of present knowledge and research. Spongiform Encephalopathy Advisory Committee, London.

6. Prusiner S. B. (1997). Prion diseases and the BSE crisis. Science 278, 245-251.

7. Stekel D. J., Nowak M. A. and Southwood T. R. (1996). Prediction of future BSE spread. Nature 381, 119.

8. Taylor D. M. (1993). Bovine spongiform encephalopathy and its association with the feeding of ruminant-derived protein. Dev. Biol. Stand 80, 215-224.

9. Meldrum K. C. (1990). Transmission of BSE to a pig. Vet. Res. 127, 362.

10. Kimberlin R. H. and Walker C. A. (1989). Pathogenesis of scrapie in mice after intragastric infection. Virus Res. 12, 213-220.

11. Fraser H., Bruce M. E., Chrec A., McConnell I. and Wells G. A. (1992). Transmission of bovine spongiform encephalopathy and scrapie to mice. J. Gen. Virol 73, 1891-1897.

12. Foster J. D., Hope J. and Fraser H. (1993). Transmission of bovine spongiform encephalopathy to sheep and goats. Vet Res. 133.

13. Culip R. C., Miller J. M., Race R. E., Jenny A. L., Katz J. B., Lehnkuhl H. D., DeBey B. M. and Robinson M. M. (1994). Intracerebral transmission of scrapie to cattle. J Infect. Dis. 169, 814-829.

14. Collinge J., Sidle K. C. L., Meads J., konside J. and Hill A. F. (1996). Molecular analysis of prion strain variation and the aetiology of new variant CJD. Nature 383, 685-690.

15. Truyen U., Parrish C. R., Harder T. C. and Kaaden O. R. (1995). There is nothing permanent except change. The emergence of new virus diseases. Vet. Microbiol. 43, 103-112.

16. Pearson G. R., Wyatt J. M., Gruffydd-Jones T. J., Hope J., Chong A., Higgins R. J., Scott A. C. and Wells G. A. (1992). Feline spongiform encephalopathy; fibril and PrP studies. Vet. Rec. 131, 307-310.

17. Fournier K. G., Escaig-Haye F., Billette de Villemeur T. and Robain 0. (1995). Ultrastructural localization of cellular prion protein (PrPc) in synaptic boutons of normal hamster hippocampus. C. R. Acad. Sci. III318, 339-334.

18. DeArmond S. J., Mobley W. C., DeMott D. L., Barry R. A., Beckstead J. H. and Prusiner S. B. (1987). Changes in the localization of brain prion protein during scrapie infection. Neurology 37, 1271-1280.

19. Hsiao K. K., Scott M., Foster D., Groth D. F., DeArmond S. J. and Prusiner S. B. (1990). Spontaneous neurodegeneration in transgenic mice with mutant prion protein. Science 250, 1587-1590.

20. Hsiao K., Scott M., Foster D., DeArmond S. J., Groth D., Serban H. and Prusiner S. B. (1991). Spontaneous neurodegeneration in transgenic mice with prion protein codon 101 proline—leucine substitution. Ann NY Acad Sci 640, 166-170.

21. Hsiao K. K., Groth D., Scott M., Yang S. L., Serban H., Rapp D., Foster D., Torchia M., Dearmond S. J. and Prusiner S. B. (1994). Serial transmission in rodents of neurodegeneration from transgenic mice expressing mutant prion protein. Proc. Nat'l Acad. Sci. USA 91, 9126-9130.

22. Liao Y. C., Lebo R. V., Clawson G. A. and Smuckler E. A. (1986). Human prion protein cDNA; molecular cloning, chromosomal mapping and biological implications. Science 233, 364-367.

23. Sparkes R. S., Simon M., Cohn V. H., Fournier R. E., Lem J., Klisak I., Heinzmann C., Blatt C., Lucero M. and Mohandas T. (1986). Assignment of the human and mouse prion protein genes to homologous chromosomes. Proc. Natl. Acad Sci. USA 83, 7358-7362.

24. Anderson R. M., Donnelly C. A., Ferguson N. M., Woolhouse M. E. J., Watt C. J., Udy H. J., MaWhinney S., Dunstan S. P., Southwood T. R. E., Wilesmith J. W., Rayns J. B. M ., Hoinville L. J., Hillerton J. E. Austin A. R. and Wells G. A. H. (1996). Transmission dynamics and epidemiology of BSE in British cattle. Nature 382, 779-788.

25. Xi Y. G., Ingrosso L., Ladogana A., Masullo C. and Pocchiari M. (1992). Amphotericin B treatment dissociates in vivo replication of the scrapie agent from PrP accumulation. Nature 356, 598-601.

26. Caughey B. and Raymond G. J. (1993). Sulfated polyanion inhibition of scrapie-associated PrP accumulation in cultured cells. J Virol 67, 643-650.

27. Bueler H., Fisher M., Lang Y., Bluethmann H., Lipp H.-P., DeArmond S. J., Prusiner S. B., Agent M. and Weissmann C. (1992). Normal development and behavior of mice lacking the neuronal cell surface PrP protein. Nature 356, 577-582.

28. Zawada W. M., Cibelli J. B., Choi P. K., Clarkson E. D., Golueke P. J., Witta S. E., Bell K. P., Kane J., Ponce de Leon F. A., Jerry J. D., Robl J. M., Freed C. R. and Stice S. L. (1998). Somatic cell cloning-produced transgenic bovine neurons for transportation in Parkinsonian rats, submitted.

29. Chisholm A. H. (1996). Fetal tissue transplantation for the treatment of Parkinson's disease: a review of the literature. J Neurosci. Nurs. 28, 329-338.

30. Defer G. L., Gerry C., Ricolfi F., Fenelon G., Monfort J. C., Remy P., Villafane G., Jeny R, Samson Y., Keravel Y., Gaston A., Degos J. D., Peschanski M., Cesaro P, and Nguyeen J. P. (1996). Long-term outcome of unilaterally transplanted Parkinsonian patients. I. Clinical approach. Brian 119, 41-50.

31. Shannon K. M. and Kordower J. H. (1996). Neural transplantation for Huntington's disease: experimental rationale and recommendations for clinical trials. Cell Transplant 5, 339-352.

32. Izraeli S., Lowe L., Bertness V., Good D. J., Kirsch I. and Kuehn M. (1998). Requirement for the immediate-early gene SIL for body axis development and Sonic Hedgehog function in mouse embryos, in preparation.

33. Good D. J., Porter F. D., Mahon K. A., Parlow A. F., Westphal H. and Kirsch I. R. (1997). Hypogonadism and obesity in mice with a targeted deletion of the Nhlh2 gene. Nat. Genet. 15, 397-401.

34. Stice S. L. and Robl J.M. (1988). Nuclear reprogramming in nuclear transplant rabbit embryos. Biol. Reprod. 39, 657-664.

35. Stice S. L., Strelchenko N. S., Keefer C. L. and Matthews L. (1996). Pluripotent bovine embryonic cell lines direct embryonic development following nuclear transfer. Biol. Reprod. 54, 100-100.

36. Sambrook J., Fritsch E. F. and Maniatis T. (1989). “Molecular Cloning: A Laboratory Manual.” Cold Spring Harbor Laboratory Press, Cold Spring Harbor.

37. Puckett C., Concannon P., Casey C. and Hood L. (1991). Genomic structure of the human prion protein gene. Am. J Hum. Genet. 49, 871-872.

38. Love P. E., Trembley M. L. and Westphal H. (1992). Targeting of the T-cell receptor zeta chain in embryonic stem cells: strategies for generating multiple mutations in a single gene. Proc. Natl Acad. Sci. USA 89, 9929-9933.

39. van Deursen J. and Wieringa B. (1992). Targeting of the creatine kinase M gene in embryonic stem cells using isogenic and nonisogenic vectors. Nucl. Acids Res. 20, 3815-3820.

40. Inoue S., Tanaka M., Horiuchi M., Ishiguro N. and Shinagawa M. (1997). Characterization of the bovine prion protein gene: the expression requires interaction between the promoter and intron. J Vet. Med. Sci. 59, 175-183.

41. Capecchi M. R. (1989). The new mouse genetics: Altering the genome by gene targeting. Trends Genet 5, 70-76.

42. Brown J. P., Wei W. and Sedivy J. M. (1997). Bypass of senescence after disruption of p. 21 cip l/wafl gene in normal diploid human fibroblasts. Science 277, 831-834.

43. Tybulewicz, V. L., Crawford C. E., Jackson P. K., Bronson R. T. and Mulligan R. C. (1991). Neonatal lethality and lymphopenia in mice with a homozygous disruption of the c-abl proto-oncogene. Cell 65, 1153-1163.

44. Mansour S. L., Thomas K. R. and Capecchi M. R. (1988). Disruption of the proto-oncogene int-2 in mouse embryo-derived stem cells; a general strategy for targeting mutations to non-selectable genes. Nature 336, 348-352.

45. Caughey B., Race R. E. and Chesebro B. (1988). Detection of prion protein mRNA in normal and scrapie-infected tissue and cell lines. J Gen. Virl. 69, 711-716.

46. Hanson K. D. and Sedivy J. M. (1995). Analysis of biological selections for high-efficiency gene targeting. Mol. Cell. Biol. 15, 45-51.

47. S. Prusiner, http://www.nmia.com/˜mdibble/prion.html

48. Proc. Natl. Acad. Sci. USA 94:13; New Scientist, Science, 17 May, p.16

49. Bueler R., Aguzzi A., Sailer A., Greiner R. A., Autenried P., Aguet M. and Weissman C. (1993). Mice devoid of PrP are resistant to scrapie. Cell 73, 1339-1347.

50. Prusiner S. B., Groth D., Serban A., Koehler R., Foster D., Torchia M., Burton D., Yang S. L. and DeArmond S. J. (1993). Ablation of the prion protein (PrP) gene in mice prevents scrapie and facilitates production of anti-PrP antibodies. Proc. Natl. Acad. Sci. USA 90,10608-10612.

51. Weissman C, Bueler R, Fischer, M, Sailer A., Aguzzi A. and Aguet M. (1994). Prp-deficient mice are resistant to scrapie. Ann. N. Y Acad, Sci., 724,235-240

What is claimed:

1. A transgenic ungulate bearing a homozygous deletion or disruption of the prion gene, wherein said deletion or disruption prevents expression of a functional endogenous prion protein, and wherein lack of expression of a functional endogenous prion protein renders said bovine unsusceptible to prion-related diseases.

2. The transgenic ungulate of claim 1, wherein said deletion or disruption is created by homologous recombination of heterologous DNA into the prion gene locus.

3. The transgenic ungulate of claim 2, wherein said heterologous DNA comprises a selectable marker.

4. The transgenic ungulate of claim 3, wherein said heterologous DNA comprises a neomycin resistance gene operably linked to a PGK promoter.

5. The transgenic ungulate of claim 3, wherein said heterologous DNA comprises a promoterless neomycin resistance gene.

6. The transgenic ungulate of claim 1, wherein said ungulate is a bovine.

7. The transgenic ungulate of claim 6, wherein said prion-related disease is bovine spongiform encephalitis (BSE).

8. The transgenic bovine of claim 7, wherein said bovine bears a heterologous gene that is extraneous to the prion gene locus.

9. The transgenic bovine of claim 8, wherein the heterologous gene is operably linked to a mammary-specific promoter, and expression of said heterologous gene enables production of a recombinant protein in the milk of said transgenic bovine.

10. An isolated DNA molecule comprising at least part of an ungulate prion gene promoter operably linked to either a selectable marker gene coding region or a reporter gene coding region.

11. The isolated DNA molecule of claim 10, wherein said molecule further comprises a second ungulate DNA sequence from or adjacent to the ungulate prion gene locus, wherein said prion promoter region and said second DNA sequence facilitate homologous recombination of said selectable marker into the ungulate genome such that said prion gene is disrupted or deleted.

12. The isolated DNA molecule of claim 10, wherein said selectable marker is a neomycin resistance gene.

13. The isolated DNA molecule of claim 12 further comprising a thymidine kinase gene.

14. A plasmid vector comprising the isolated DNA molecule of claim 10.

15. A DNA targeting molecule capable of specifically and functionally deleting or disrupting expression of an ungulate prion gene, wherein said disruption occurs by homologous recombination into said ungulate prion gene locus.

16. The DNA targeting molecule of claim 15, wherein the targeting molecule facilitates the deletion or disruption of exon 3 of the prion gene.

17. The DNA targeting molecule of claim 15, wherein said targeting molecule comprises a selectable marker gene.

18. The DNA targeting molecule of claim 17, wherein said selectable marker gene is a neomycin resistance gene.

19. The DNA targeting molecule of claim 18, further comprising a thymidine kinase gene.

20. A plasmid vector comprising the DNA targeting molecule of claim 19.

21. The plasmid vector of claim 20, wherein said ungulate prion gene is the bovine prion gene.

22. A cloned transgenic ungulate having the same genotype as the transgenic ungulate of claim 1, wherein said cloned transgenic ungulate is created using nuclear transfer techniques.

23. The cloned transgenic ungulate of claim 22, wherein said ungulate bears a heterologous gene that is extraneous to the prion gene locus.

24. The transgenic ungulate of claim 23, wherein said ungulate is a bovine.

25. The transgenic ungulate of claim 24, wherein the heterologous gene is operably linked to a mammary-specific promoter, and expression of said heterologous gene enables production of a therapeutic protein in the milk of said transgenic bovine.

26. A line of transgenic ungulates having the same genotype as the transgenic ungulate of claim 1.

27. A line of transgenic ungulates having the same genotype as the cloned transgenic ungulate of claim 22.

28. A transgenic ungulate comprising a targeted gene deletion.

29. A transgenic bovine comprising a targeted gene deletion.

30. A transgenic ungulate bearing a heterozygous deletion of the prion gene, wherein said deletion prevents expression of a functional endogenous prion protein from one prion gene allele, and wherein lack of expression of a functional endogenous prion protein from said one allele renders said ungulate less susceptible to prion-related diseases.

31. A method of making the transgenic ungulate of claim 30, comprising the steps of:
(1) isolating genomic DNA from ungulate cells;
(2) isolating a prion gene allele from said genomic DNA;
(3) determining a restriction enzyme map and the intron/exon structure of the ungulate prion gene allele isolated from said ungulate genomic DNA;
(4) sub-cloning fragments from said prion gene allele for the construction of a targeting DNA molecule;
(5) constructing a targeting DNA molecule which is capable of disrupting or deleting an ungulate prion gene allele by homologous recombination;
(6) transfecting said ungulate cells such that homologous recombinants are isolated;
(7) transferring the nuclei from a transfected cell containing the targeting molecule homologously recombined into a prion gene allele to the cytoplasm of an enucleated mature ungulate oocyte;
(8) culturing said oocyte to form a blastocyst; and
(9) transferring said blastocyst to a recipient ungulate such that a transgenic ungulate according to claim 30 is born.

32. A method of making the transgenic ungulate of claim 1, comprising the steps of:
(1) isolating genomic DNA from ungulate cells;
(2) isolating a prion gene allele from said genomic DNA;
(3) determining a restriction enzyme map and the intron/exon structure of the bovine prion gene allele isolated from said ungulate genomic DNA;
(4) sub-cloning fragments from said prion gene allele for the construction of a targeting DNA molecule;
(5) constructing a targeting DNA molecule which is capable of disrupting or deleting an ungulate prion gene allele by homologous recombination;
(6) transfecting said ungulate cells such that homologous recombinants are isolated;
(7) transferring the nuclei from a transfected cell containing the targeting molecule homologously recombined into a prion gene allele to the cytoplasm of an enucleated mature ungulate oocyte;
(8) culturing said oocyte to form a blastocyst;
(9) transferring said blastocyst to a recipient ungulate such that a transgenic ungulate having a heterozygous deletion or disruption in the prion gene is born; and
(10) breeding said transgenic ungulate having a heterozygous deletion or disruption in the prion gene to obtain the transgenic ungulate of claim 1, or targeting a deletion of the other allele using primary fibroblasts derived from a heterozygous transgenic ungulate to create the transgenic ungulate of claim 1.

33. The method of claim 31, wherein said ungulate cells are bovine cells.

34. The method of claim 32, wherein said ungulate cells are bovine cells.

35. The method of claim 33, wherein said bovine cells are derived from fetal fibroblast cells.

36. The method of claim 34, wherein said bovine cells are derived from fetal fibroblast cells.

37. The method of claim 35, wherein said cells are BFF cells.

38. The method of claim 36, wherein said cells are BFF cells.

39. The transgenic ungulate of claim 1, wherein said ungulate bears a heterologous gene that is extraneous to the prion gene locus.

40. The transgenic ungulate of claim 39, wherein said extraneous heterologous gene is a mutant prion gene allele which confers an increased tendency to develop a prion-related spongiform encephalopy.

41. The cloned transgenic ungulate of claim 23, wherein said extraneous heterologous gene is a mutant prion gene allele which confers an increased tendency to develop a prion-related spongiform encephalopy.

42. A method of using the transgenic ungulate of claim 40 to screen for or evaluate agents which may be used in the treatment or prevention of spongiform encephalopathies, said method comprising:
(1) administering a putative therapeutic agent to said transgenic ungulate before or after the development of said prion-related spongiform encephalopy; and
(2) monitoring said ungulate to determine whether said prion-related spongiform encephalopy has been prevented or treated.

43. A method of xenotransplantation using fetal tissue or cells derived from the transgenic ungulate of claim 1, said method comprising:
(1) generating a transgenic fetus having the same genotype as the transgenic ungulate of claim 1, either by mating or cloning techniques;
(2) isolating tissue or cells of interest from said fetus; and
(3) transplanting the fetal tissue or cells into a recipient mammal.

44. The method of claim 43, wherein said cells are fetal neurons.

45. The method of claim 44, wherein said cells are derived from fetal corneal tissue.

46. The transgenic ungulate of claim 1, wherein said ungulate bears at least one other deletion or disruption that is extraneous to the prion gene locus.

47. The transgenic bovine of claim 46, wherein said at least one other deletion or disruption is in a gene which interferes with xenotransplantation.

48. A method of xenotransplantation using fetal tissue or cells derived from the transgenic bovine of claim 44, said method comprising:
(1) generating a transgenic fetus having the same genotype as the transgenic bovine of claim 1, either by mating or cloning techniques;
(2) isolating tissue or cells of interest from said fetus; and
(3) transplanting the fetal tissue or cells into a recipient mammal.

49. A method of using the transgenic bovine of claim 8 for the production of recombinant proteins, said method comprising:
(1) generating a female transgenic bovine according to claim 8; and
(2) isolating the recombinant protein from the milk of said transgenic bovine.
